SEQUENCE LISTING (2 sequences)

SEQ ID NO: 1 - protein, 385 aa, Homo sapiens
  isoform A of SMARCB1 (1)..(385)
MMMMALSKTFGQKPVKFQLEDDGEFYMIGSEVGNYLRMFRGSLYKRYPSLWRRLATVEERKKIVASSHGKKTKPNTKDHGYTTLATSVTLLKASEVEEILDGNDEKYKAVSISTEPPTYLREQKAKRNSQWVPTLPNSSHHLDAVPCSTTINRNRMGRDKKRTFPLCFDDHDPAVIHENASQPEVLVPIRLDMEIDGQKLRDAFTWNMNEKLMTPEMFSEILCDDLDLNPLTFVPAIASAIRQQIESYPTDSILEDQSDQRVIIKLNIHVGNISLVDQFEWDMSEKENSPEKFALKLCSELGLGGEFVTTIAYSIRGQLSWHQKTYAFSENPLPTVEIAIRNTGDADQWCPLLETLTDAEMEKKIRDQDRNTRRMRRLANTAPAW

SEQ ID NO: 2 - protein, 376 aa, Homo sapiens
  isoform B of SMARCB1 (1)..(376)
MMMMALSKTFGQKPVKFQLEDDGEFYMIGSEVGNYLRMFRGSLYKRYPSLWRRLATVEERKKIVASSHDHGYTTLATSVTLLKASEVEEILDGNDEKYKAVSISTEPPTYLREQKAKRNSQWVPTLPNSSHHLDAVPCSTTINRNRMGRDKKRTFPLCFDDHDPAVIHENASQPEVLVPIRLDMEIDGQKLRDAFTWNMNEKLMTPEMFSEILCDDLDLNPLTFVPAIASAIRQQIESYPTDSILEDQSDQRVIIKLNIHVGNISLVDQFEWDMSEKENSPEKFALKLCSELGLGGEFVTTIAYSIRGQLSWHQKTYAFSENPLPTVEIAIRNTGDADQWCPLLETLTDAEMEKKIRDQDRNTRRMRRLANTAPAW

(19) United States
(12) Patent Application Publication
(10) Pub. No.: US 2022/0218708 A1
(43) Pub. Date: Jul. 14, 2022

(54) METHODS FOR TREATING SMARCB1 DEFICIENT CANCER OR PAZOPANIB RESISTANT CANCER

(71) Applicant: The Institute of Cancer Research:Royal Cancer Hospital, London (GB)

(72) Inventors: Paul Huang, London (GB); Jocelyn Wong, London (GB)

(21) Appl. No.: 16/304,935

(22) PCT Filed: May 26, 2017
(86) PCT No.: PCT/EP2017/062792

§ 371 Date: Mar. 2, 2022

(30) Foreign Application Priority Data
May 27, 2016 (GB) 1609402.1

(51) Int. Cl. A61K 31/5025; A61K 31/47; A61K 31/506

(52) U.S. Cl. CPC A61K 31/5025; A61K 31/506; A61K 31/47

(57) ABSTRACT

The present invention provides an inhibitor of PDGFRα and an inhibitor of FGFR for use in a method of treating a SMARCB1 deficient cancer in an individual. Also provided is an FGFR inhibitor for use in a method of sensitizing cancer cells to a PDGFRα inhibitor in the treatment of a SMARCB1 deficient cancer, by administering an FGFR1 inhibitor and a PDGFRα inhibitor.

### FIELD OF THE INVENTION

The present invention relates to materials and methods for treating SMARCB1 deficient cancers and to materials and methods of treating cancers that are resistant to pazopanib.

### BACKGROUND

Inactivating mutations in genes encoding components of the SWI/SNF chromatin remodelling complex are found in ˜20% of cancers (Kadoch et al., 2013). Treatment of this class of tumours is challenging and there are currently no targeted therapies approved for clinical use. The prototypical example of this class is the malignant rhabdoid tumours (MRTs) which are rare and lethal paediatric cancers of the kidney and soft tissues.

MRTs are highly aggressive and despite intensive multimodal therapy, prognosis remains dismal with many children not surviving beyond 12 months (Madigan et al., 2007). Many MRT patients are refractory to standard chemotherapy and are often lethal within the first year of diagnosis (Madigan et al., 2007). There is thus an urgent need for new effective therapies.

MRTs are characterised by the bi-allelic inactivation of the SMARCB1 (INI1/SNFS/BAF47) gene which encodes a core component of the SWI/SNF complex and is a tumour suppressor (Kim and Roberts, 2014). SMARCB1 mutation is the sole driver of disease and MRTs lack additional gene amplifications or deletions and demonstrate low rates of mutations (Lee et al., 2012). The mechanisms by which SMARCB1 loss contributes to tumour progression are not fully understood and analyses of genes regulated by SMARCB1 have revealed several candidate oncogenes, including components of the cell cycle machinery, sonic hedgehog pathway and canonical Wnt signalling (Kim and Roberts, 2014). Identifying the fundamental oncogenic drivers resulting from SMARCB1 deficiency remains a significant challenge and a key barrier to developing effective therapies.

Pazopanib is used in the treatment of renal cell carcinoma and soft tissue sarcoma. However, resistance to pazopanib develops in all patients treated (Kasper et al. 2014). There is therefore a need to find suitable treatments for patients who have developed resistance to pazopanib.

### SUMMARY OF THE INVENTION

In one aspect the present invention is based on research to identify oncogenic drivers in SMARCB1 deficient cancers such as malignant rhabdoid tumours (MRT). In doing so, the inventors found that dual inhibition of two targets, PDGFRalpha and FGFR has synergistic efficacy. In particular, the inventors show that while inhibition of each target singly does not induce apoptosis of the target cell, dual inhibition results in synergistic cytotoxicity in cells with SMARCB1 deficiency. The present inventors also show that treatment with FGFR inhibitors sensitizes MRT cells that have acquired resistance to a PDGFRα inhibitor.

Previous reports found that A204 cells are sensitive to sunitinib and dasatinib (albeit mislabelled as a rhabdomyosarcoma line) through inhibition of PDGFRα (Bai et al., 2012; McDermott et al., 2009). Separately, The FGFR inhibitor BGJ398 has also been shown to reduce MRT cell growth (Wohrle et al., 2013). However, the inventor's experiments demonstrate that these inhibitors have limited utility as single agents and do not induce apoptosis.

The inventors have shown that PDGFRα levels are regulated by SMARCB1 expression. An integrated molecular profiling and chemical biology approach demonstrated that the receptor tyrosine kinases (RTKs) PDGFRα and FGFR1 are co-activated in MRT cells.

The inventors have demonstrated for the first time that dual inhibition/blockade of PDGFRα and FGFR leads to suppression of AKT and ERK1/2 phosphorylation resulting in synergistic cytotoxicity in MRT cells.

Accordingly, in a first aspect, the present invention relates to methods of treatment SMARCB1 deficient cancer in an individual, the method involving inhibition of both FGFR and PDGFRα.

The invention provides an inhibitor of PDGFRα and an inhibitor of FGFR for use in a method of treating an individual having SMARCB1 deficient cancer. Put another way, the invention provides one or more receptor tyrosine kinase inhibitors for use in a method of treating an individual having SMARCB1 deficient cancer, wherein the receptor tyrosine kinase inhibitor(s) collectively inhibit PDGFRα and FGFR.

Cancers which can be treated according to the first aspect of the invention include:

rhabdoid tumours including malignant rhabdoid tumours (MRT) and atypical teratoid rhabdoid tumours (AT/RT), epithelioid sarcoma, renal medullary carcinoma, epithelioid malignant peripheral nerve sheath tumour, extraskeletal myxoid chondrosarcoma, cribiform neuroepithelial tumour of the ventricle, collecting duct carcinoma and synovial sarcomas. The cancer to be treated may be a rhabdoid tumour, for example MRT.

The inhibitors for use in the invention may be any of a small molecule inhibitor, an antibody, a ligand trap, a peptide fragment and a nucleic acid inhibitor. The PDGFRα inhibitor and the FGFR inhibitor may be the same type of inhibitor (e.g. both small molecule inhibitors) or they may be different.

The PDGFRα inhibitor may be selected from pazopanib, ponatinib, dasatinib, olaratumab, lucitanib and sunitinib.

The FGFR inhibitor may be an inhibitor of FGFR1, FGFR2, FGFR3 and/or FGFR4. In some embodiments the products and uses of the invention involve inhibition of FGFR1, 2 and/or 3. The products and uses of the invention may involve inhibition of FGFR1. In other words the FGFR inhibitor may be an FGFR1 inhibitor. The term FGFR1 inhibitor does not exclude inhibition by that inhibitor of other FGFRs.

In practice many inhibitors inhibit multiple FGFRs (Patani et al. 2016), and so administration of an inhibitor according to the invention may inhibit multiple FGFRs. The FGFR inhibitor may be selected from NVP-BGJ398, AZD4547, TKI258, JNJ42756493, lucitanib and ponatinib. These inhibitors are all FGFR1 inhibitors.

The inhibitor of PDGFRα and inhibitor of FGFR may be the same molecule. In other words a dual inhibitor of PDGFRα and FGFR may be used. For example, the inhibitor may be ponatinib or lucitanib. For example, the inhibitor may be ponatinib. The PDGFRα and FGFR inhibitors may be different.

One or more inhibitors of PDGFRα or FGFR may be used for treatment according to the invention in combination with a dual-inhibitor of PDGFRα and FGFR.

In the methods and uses, the PDGFRα and FGFR inhibitors may have a synergistic effect. Accordingly, the inhibitors provided herein may be for use in a method of providing synergistic activity in the treatment of cancer.

In the methods and uses, the combination and the PDGFRα inhibitor and the FGFR inhibitor, e.g. FGFR1 inhibitor, may result in apoptosis of the cancer cells. Accordingly the inhibitors provided may be for use in a method of inducing apoptosis in the treatment of cancer.

The inventors have shown that FGFR inhibitors can be used to sensitize cells to PDGFRα inhibitors, which have acquired resistance to PDGFRα inhibitors. In the methods and uses the combination of inhibitors can be used to treat cancer that is resistant to treatment with a PDGFRα inhibitor alone. The cancer may have reduced expression of PDGFRα or may not express PDGFRα.

The invention provides an FGFR inhibitor for use in a method of sensitizing cells to a PDGFRα inhibitor in the treatment of cancer, the method comprising administration of the FGFR inhibitor and PDGFRα inhibitor.

It is envisaged that the combination of an FGFR inhibitor and a PDGFRα inhibitor can be used to treat an individual who has a cancer with acquired resistance to PDGFRα inhibitor. An FGFR inhibitor and a PDGFRα inhibitor are therefore provided for use in a method of treating a SMARCB1 deficient cancer in an individual, wherein the cancer has acquired resistance to a PDGFRα inhibitor. The PDGFRα inhibitor used in the combined treatment may be the same as the inhibitor to which the cancer has acquired resistance.

In connection with the treatment of SMARCB1 deficient cancers the combination of inhibitors may be used in a method of sensitising PDGFRα inhibitor resistant cancer, increasing sensitivity of cancer cells to PDGFRα inhibitors, prolonging sensitivity to PDGFRα inhibitors and/or preventing/inhibiting acquired drug resistance to PDGFRα inhibitors.

Accordingly, in the methods and uses, the combination of inhibitors may be used to prevent acquired drug resistance to a PDGFRα inhibitor. The combination of inhibitors may be used to delay the onset of acquired drug resistance to a PDGFRα inhibitor.

The inhibitor of PDGFRα and the inhibitor of FGFR may be administered simultaneously or sequentially. In some embodiments the inhibitors are in the same composition.

The methods and uses may comprise the step of determining whether the individual has SMARCB1 deficient cancer. This may be by determining SMARCB1 protein expression in a sample obtained from the individual, for example using immunohistochemistry.

This aspect of the invention may also be defined as the use of an inhibitor of PDGFRα and an inhibitor of FGFR in the manufacture of a medicament for the treatment of a SMARCB1 deficient cancer, or as a method of treating a SMARCB1 deficient cancer in an individual, the method comprising administering a therapeutically effective amount of an inhibitor of PDGFRα and an inhibitor of FGFR to the individual.

The invention provides a pharmaceutical composition comprising an inhibitor of PDGFRα and an inhibitor of FGFR, wherein the inhibitor of PDGFRα and the inhibitor of FGFR are different.

Acquired resistance and tumour recurrence is common in patients undergoing tyrosine kinase inhibitor (TKI) therapy. Pazopanib is approved for sarcoma treatment but patients eventually develop resistance by mechanisms that are unknown (Kasper et al., 2014). The inventors probed a number of cell lines for sensitivity for pazopanib (FIG. 1A, middle graph; table S1, third column), and identified two cell lines which were sensitive. These lines were used to generate an acquired resistance model (FIG. 1B, middle graph; table S2). The inventors present the first mechanism of acquired resistance to pazopanib in soft tissue malignancies through PDGFRα loss and provides a means to overcome this resistance via FGFR blockade. This mechanism is not binding on the methods of the invention.

Accordingly, in a second aspect the present invention is based on the findings of a treatment for pazopanib resistant cancers. In this aspect the invention relates to methods of treatment of pazopanib resistant cancers in an individual, the method involving inhibition of FGFR, for example FGFR1.

The invention provides an FGFR inhibitor for use in a method of treating a pazopanib resistant cancer in an individual.

The pazopanib resistant cancer may be a renal cell carcinoma or a soft tissue sarcoma, which are both types of cancer that are treated with pazopanib. For example, the pazopanib resistant cancer may be a soft tissue sarcoma.

The FGFR inhibitor may be a small molecule inhibitor, an antibody, a ligand trap, a peptide fragment or a nucleic acid inhibitor. The FGFR inhibitor may be an inhibitor of FGFR1, FGFR2, FGFR3 and/or FGFR4, for example, an inhibitor of FGFR1 FGFR2 and/or FGFR3. In particular, the FGFR inhibitor may be an inhibitor of FGFR1.

The FGFR inhibitor may be selected from NVP-BGJ398, AZD4547, TKI258, JNJ42756493, lucitanib and ponatinib.

The treatments of pazopanib resistant cancer may also involve administering a PDGFRα inhibitor. The PDGFRα inhibitor may be any one of those described herein.

There are a number of methods to determine resistance of a tumour to pazopanib treatment. For example, resistance to pazopanib may be determined by tumour growth and/or metastasis after treatment with pazopanib. Accordingly, the methods and uses may involve the step of determining that the cancer is pazopanib resistant and selecting the individual having pazopanib resistant cancer for treatment.

The determining step may comprise imaging the individual to determine tumour size and/or detect metastasis. For example, the individual may be imaged a plurality of times over the course of treatment with pazopanib.

The methods and uses may also comprise the step of determining FGFR expression. FGFR expression can be determined by a number of methods as described elsewhere herein, for example, in a sample of cancer cells obtained from the individual. Where the cancer expresses FGFR, the individual can be selected for treatment with an FGFR inhibitor. Accordingly the cancer to be treated may express FGFR, e.g. FGFR protein.

This aspect of the invention also provides the use of an inhibitor of FGFR in the manufacture of a medicament for the treatment of pazopanib resistant cancer in an individual. Also provided is a method of treating pazopanib resistant cancer in an individual, the method comprising administering to the individual a therapeutically effective amount of an inhibitor of FGFR.

### FIGURES

FIG. 1. MRT cell lines are sensitive to PDGFRα inhibitors. (A) Dose response curves of dasatinib, pazopanib and sunitinib resistant (black) and sensitive (red) cell lines. A panel of 14 cell lines were treated with a range of drug concentrations to determine IC₅₀ values (Table S1). Cell viability is normalised to DMSO control (n=2 or 3). (B) Dose response curves of TKI resistant sublines (black) and parental A204 cells (red), IC₅₀ values are detailed in Table S2. Cell viability is normalised to DMSO control (n=3). (C) Target selectivity overlap plot of dasatinib, pazopanib and sunitinib shows that KIT, CSF1R and PDGFRA are common targets. (D) Immunoblot of PDGFRα expression in parental A204 and resistant sublines. DasR=dasatinib resistant, PazR=pazopanib resistant and SunR=sunitinib resistant. (E) Immunoprecipitation of PDGFRα followed by immunoblotting with phosphotyrosine-specific antibody (PY1000) shows a decrease in receptor phosphorylation with 1 μM TKI for 1 hour. (F) Immunoblot of PDGFRα expression in the MRT cells under mock, non-targeting control siCONT and siPDGFRα pool transfection conditions. (G) Bar plots showing cell viability of MRT cells upon siRNA silencing of PDGFRα. Cell viability data is normalised to mock transfection (n=3). Statistical analysis of siPDGFRα versus siCONT was performed by paired Student's t test where *p<0.05. (H) Immunoblot of AKT and ERK1/2 phosphorylation levels treated with TKIs at the indicated doses for 3 hours. (I) Immunoblot of PDGFRα showing downregulation of receptor levels upon ectopic SMARCB1 expression. For (A), (B) & (G), all values are mean±SD.

FIG. 2. Molecular profiling of A204 cells. (A) aCGH plots of A204 parental and resistant cells. Selected profiles of chromosome 22 illustrating focal deletion of SMARCB1 in 22q11.23. DasR harbours chromosome 17 and 13 alterations illustrating gains (green) and losses (red) respectively. Full genomic profiles are presented in FIG. 5A. (B) Heatmap of the top 50 downregulated genes in the resistant sublines versus the parental A204 cells treated with TKIs. Full gene expression dataset is presented in FIG. 5B. (C) Heatmap of phosphoproteomic data with log₂ fold change of untreated A204 parental cells versus DasR or PazR in the presence of TKI versus with PDGFRα and FGFR1 phosphorylation sites highlighted in red and blue respectively. Grey boxes represent phosphosites that were not observed under that specific condition. Data presented is an average of three independent experiments.

FIG. 3. Dual inhibition of PDGFRα and FGFR1 is cytotoxic in MRT cells. (A) Dose response curves for MRT and AN3CA cell lines upon treatment with FGFR inhibitors BGJ398 and AZD4547. Cell viability is normalised to DMSO control (n=3). (B) Immunoblot of FGFR1 expression in MRT cells under mock, non-targeting control siCONT and siFGFR1 pool transfection conditions. (C) Bar plots showing cell viability of MRT cells upon siRNA silencing of FGFR1. Cell viability data is normalised to mock transfection (n=3). Statistical analysis of siFGFR1 versus siCONT was performed by paired Student's t test where **p<0.01 and NS is not significant. (D) Bar plots showing the normalised fold change in caspase 3/7 activity in the A204 cells treated with PDGFRα and FGFR inhibitors or a combination at the indicated doses (n=3). Data for G402 cells are presented in FIG. 6B. Data is normalised to DMSO control. Statistical analysis between combination and single TKI treatment was done by ANOVA with Tukey's multiple comparison test where ***p<0.001. (E) Combination index measurements for BGJ398 and PDGFRα inhibitors in A204 cells show synergy (CI<1) across all doses tested. Individual dose response measurements are presented in FIG. 6D. (F) Dose response curves of ponatinib resistant (black) and sensitive (red) cell lines. A panel of 14 cell lines were treated with a range of ponatinib concentrations. Cell viability is normalised to DMSO control (n=2). (G) Bar plots showing the normalised fold change in caspase 3/7 activity in the A204 and G402 cells treated with ponatinib (n=3). Data is normalised to DMSO control. Statistical analysis performed by paired Student's t test where *p<0.05. (H) Immunoblot of AKT and ERK1/2 phosphorylation levels upon drug treatment at the indicated doses for 1 hour. (I) Dose response curves for PazR cells treated with pazopanib, BGJ398, a combination of both or ponatinib. Cell viability is normalised to DMSO control (n=3), IC₅₀ values are detailed in Table S3. (J) Bar plots showing percentage annexin V staining in PazR cells treated with pazopanib, BGJ398, a combination of both inhibitors or ponatinib (n=3). Statistical analysis of TKI treatment versus DMSO was done by paired Student's t test where *p<0.05 and **p<0.01 and NS is not significant. Data presented for (A), (C), (D), (F), (G), (I) and (J) are means±SD.

FIG. 4. Colony formation assay showing that pazopanib treatment over 2 weeks leads to resistant colony formation in the A204 cells. Treatment with high dose combination of pazopanib and AZD4547 led to no colonies, providing support that first line combination therapy prevents acquisition of resistance.

FIG. 5. (A) Microarray-based comparative genomic hybridisation plots of A204 parental and resistant cells displaying the full genomic profiles of the four cell lines. (B) Hierarchical clustering of gene expression dataset of parental A204 cells treated with DMSO control or each of the three PDGFRAα TKIs and each resistant subline treated with their respective TKI. DasR=dasatinib resistant, PazR=pazopanib resistant and SunR=sunitinib resistant.

FIG. 6. Dual inhibition of PDGFRα and FGFR1 is cytotoxic in MRT cells. (A) Dose response curves for A204 and G402 cells upon treatment with PDGFRα and a combination of PDGFRα and FGFR inhibitors. Cell viability data is normalised to DMSO control (n=3). Values are mean±SD. (B) Bar plots showing the normalised fold change in caspase 3/7 activity in the G402 cells upon treatment with PDGFRα and FGFR inhibitors or a combination at the indicated doses (n=3). Data is normalised to DMSO control. Statistical significance of combination versus single TKI treatment was performed by ANOVA with Tukey's multiple comparisons test where ***p<0.001. (C) Bar plots showing apoptosis measured by caspase 3/7 activity (left) and viability (right) of A204 cells treated with FGFR inhibitors in combination with siRNA depletion of PDGFRα. Statistical analysis of FGFR inhibitors versus DMSO control was performed by paired Student's t test where *p<0.05. Values are mean±SD. (D) Bar plots showing percentage Annexin V staining in A204 parental cells when treated with PDGFRα inhibitor, BGJ398 or a combination of both inhibitors (n=3). Data presented is means±SD. (E) Bar plots showing percentage Annexin V staining in A204 parental cells treated with ponatinib (n=3). Data presented is mean±SD. (F) Immunoprecipitation of PDGFRα followed by immunoblotting with phosphotyrosine-specific antibody (PY1000) in A204 cells upon treatment with 1 μM PDGFRα inhibitor, BGJ398, combination or ponatinib for 1 hour.

FIG. 7. Targeting FGFR1 sensitizes acquired resistance to pazopanib. (A) Immunoblot of FGFR1 expression in the parental A204 and resistant sublines. DasR=dasatinib resistant, PazR=pazopanib resistant and SunR=sunitinib resistant. (B) Representative images of dual-colour immunofluorescence analysis of parental A204 and resistant sublines, DAPI (blue), FGFR1 (red) and PDGFRα (green) showing that FGFR1 and PDGFRα expression is uniformly distributed in all cells within the parental A204 population.

### DETAILED DESCRIPTION

Receptor tyrosine kinases (RTKs) are attractive targets for cancer therapy, with several tyrosine kinase inhibitors (TKIs) clinically approved for a range of tumour types (Lemmon and Schlessinger, 2010).

Cancer cells rely on the activation of multiple RTKs to maintain robust oncogenic signalling (Huang et al., 2007), and employing TKI combinations is effective in overcoming compensatory RTK signalling and ultimately killing cancer cells (Xu and Huang, 2010). However, the mechanisms by which SMARCB1 loss contributes to tumour progression were not fully understood.

The present inventors have found that MRT cells display coactivation of PDGFRα and FGFR and that therapeutic inhibition of both RTKs leads to synergistic cytotoxicity.

In the present invention, references to PDGFRα denote the receptor tyrosine kinase (RTK) platelet-derived growth factor alpha. PDGFRα is a cell surface tyrosine kinase receptor.

The HUGO Gene Symbol report for PDGFRα can be found at: http://www.genenames.org/cgi-bin/gene symbol report?hgnc id=8803 which provides links to the human PDGFRα nucleic acid and amino acid sequences, as well as reference to the homologous murine and rat proteins. The human form has the HGNC ID: 8803, and the ensemble gene reference ENSG00000134853. The uniprot reference is P16234.

References to FGFR denote the family of receptor tyrosine kinase (RTK) fibroblast growth factor receptors, including FGFR1, FGFR2, FGFR3 and FGFR4. FGFRs are cell surface tyrosine kinase receptors. Thus, reference to the expression or inhibition of FGFR refers to expression of inhibition of at least one of the FGFR family, for example FGFR1, FGFR2, FGFR3 and/or FGFR4, for example, at least FGFR1.

The HUGO Gene Symbol report for FGFR1 can be found at: http://www.genenames.org/cgi-bin/gene symbol report?hgnc id=HGNC:3688 which provides links to the human FGFR1 nucleic acid and amino acid sequences, as well as reference to the homologous murine and rat proteins. The human form has the HGNC ID: 3688, and the ensemble gene reference ENSG00000077782. The uniprot reference is P11362.

The HUGO Gene Symbol report for FGFR2 can be found at: http://www.genenames.org/cgi-bin/gene symbol report?hgnc id=HGNC:3689 which provides links to the human FGFR2 nucleic acid and amino acid sequences, as well as reference to the homologous murine and rat proteins.

The human form has the HGNC ID: 3689, and the ensemble gene reference ENSG00000066468. The uniprot reference is P21802.

The HUGO Gene Symbol report for FGFR3 can be found at: http://www.genenames.org/cgi-bin/gene symbol report?hgnc id=HGNC:3690 which provides links to the human FGFR3 nucleic acid and amino acid sequences, as well as reference to the homologous murine and rat proteins. The human form has the HGNC ID: 3690, and the ensemble gene reference ENSG00000068078. The uniprot reference is P22607.

The HUGO Gene Symbol report for FGFR4 can be found at: http://www.genenames.org/cgi-bin/gene symbol report?hgnc id=HGNC:3691 which provides links to the human FGFR4 nucleic acid and amino acid sequences, as well as reference to the homologous murine and rat proteins. The human form has the HGNC ID: 3691, and the ensemble gene reference ENSG00000160867. The uniprot reference is P22455.

References to SMARCB1 denote SWI/SNF related, matrix associated, actin dependent regulator of chromatin, subfamily b, member 1. Reference to SMARCB1 can refer to any isoform of the protein.

The HUGO Gene Symbol report for SMARCB1 can be found at: http://www.genenames.org/cgi-bin/gene symbol report?hgnc id=HGNC:11103 which provides links to the human SMARCB1 nucleic acid and amino acid sequences, as well as reference to the homologous murine and rat proteins. The human form has the HGNC ID: 11103, and the ensemble gene reference ENSG00000099956. The uniprot reference is Q12824.

The amino acid sequence for human isoform A of SMARCB1 (SEQ ID NO: 1) is:

MMMMALSKTFGQKPVKFQLEDDGEFYMIGSEVGNYLRMFRGSLYKRYPSL
WRRLATVEERKKIVASSHGKKTKPNTKDHGYTTLATSVTLLKASEVEEIL
DGNDEKYKAVSISTEPPTYLREQKAKRNSQWVPTLPNSSHHLDAVPCSTT
INRNRMGRDKKRTFPLCFDDHDPAVIHENASQPEVLVPIRLDMEIDGQKL
RDAFTWNMNEKLMTPEMFSEILCDDLDLNPLTFVPAIASAIRQQIESYPT
DSILEDQSDQRVIIKLNIHVGNISLVDQFEWDMSEKENSPEKFALKLCSE
LGLGGEFVTTIAYSIRGQLSWHQKTYAFSENPLPTVEIAIRNTGDADQWC
PLLETLTDAEMEKKIRDQDRNTRRMRRLANTAPAW

The amino acid sequence for human isoform B of SMARCB1 (SEQ ID NO: 2) is:

MMMMALSKTFGQKPVKFQLEDDGEFYMIGSEVGNYLRMFRGSLYKRYPSL
WRRLATVEERKKIVASSHDHGYTTLATSVTLLKASEVEEILDGNDEKYKA
VSISTEPPTYLREQKAKRNSQWVPTLPNSSHHLDAVPCSTTINRNRMGRD
KKRTFPLCFDDHDPAVIHENASQPEVLVPIRLDMEIDGQKLRDAFTWNMN
EKLMTPEMFSEILCDDLDLNPLTFVPAIASAIRQQIESYPTDSILEDQSD
QRVIIKLNIHVGNISLVDQFEWDMSEKENSPEKFALKLCSELGLGGEFVT
TIAYSIRGQLSWHQKTYAFSENPLPTVEIAIRNTGDADQWCPLLETLTDA
EMEKKIRDQDRNTRRMRRLANTAPAW

Inhibitors of FGFR and/or PDGFRα

Compounds which may be employed for use in the present invention for treating SMARCB1 deficient cancer are receptor tyrosine kinase inhibitors, more specifically inhibitors of PDGFRα and/or FGFR.

In the context of treating SMARCB1 deficient cancer, reference to inhibitors of “PDGFRα and/or FGFR” reflects that, while the invention relates to treatment involving inhibition of both of these RTKs, the methods of treatment may involve use of a dual inhibitor of PDGFRα and FGFR, or an inhibitor of PDGFRα and an inhibitor of FGFR that are not the same molecule. In other words the PDGFRα and FGFR inhibitors may be different.

In the second aspect of the invention, relating to the treatment of pazopanib resistant cancer, treatment with an FGFR inhibitor alone is sufficient, and the FGFR inhibitors described below may be used in this context.

In some embodiments PDGFRα inhibitors may also be used alongside FGFR inhibitors for treating pazopanib resistant cancers. In these instances, the PDGFR inhibitors described herein may be used.

The term “PDGFRα inhibitor” and “inhibitor of PDGFRα” are equivalent. Likewise, the terms “FGFR inhibitor” and “inhibitor of FGFR” may be used interchangeably.

An inhibitor for use in the invention may be dual inhibitor of PDGFRα and FGFR. Alternatively, different inhibitors for PDGFRα and FGFR may be employed. The invention may make use of a plurality of inhibitors. The inhibitors may be selective for PDGFRα or FGFR.

Inhibitors of PDGFRα and/or FGFR are known in the art and are characterised by significantly inhibiting the kinase activity of PDGFRα and/or FGFR, or specifically decreasing the about of such kinase activity in cells. Exemplary inhibitors include small molecule inhibitors, antibodies, ligand traps, peptide fragments and nucleic acid inhibitors, such as siRNA and antisense molecule targeting FGFR or PDGFRα RNA.

The inhibitors may be used in a therapeutically effective amount. In the context of the treatment of SMARCB1 deficient cancers, the inhibitors may be used in an amount which allows synergistic activity between the two inhibitors and/or induces apoptosis of cancer cells and/or induces sensitivity to PDGRFα inhibitors (that have acquired resistance) and/or inhibits resistance to a PDGFRα inhibitors.

Although a “PDGFRα inhibitor” is referred to herein, in practice, many inhibitors of PDGFRα will also inhibit the beta isoform (PDGFRβ). Inhibition of the beta isoform is also envisioned as part of the invention.

Inhibitors of these receptor tyrosine kinases may interfere with expression of the receptor, with ligand binding, with receptor dimerization or with the catalytic domain, for example.

### Small Molecule Inhibitors

An inhibitor for use in the invention may be a small molecule inhibitor. Small molecule inhibitors of PDGFRα and/or FGFR are already known to the skilled person, and further suitable small molecule inhibitors may be identified by the use of high throughput screening strategies.

In one aspect a small molecule dual inhibitor of PDGFRα and FGFR may be used. For example, the dual inhibitor may be ponatinib or a pharmaceutically acceptable salt thereof.

Ponatinib is disclosed, for example, in WO2007/075869 and WO2011/053938, and has the CAS Registry No. 943319-70-8 and formal name 3-(2-imidazo[1,2-b]pyridazin-3-ylethynyl)-4-methyl-N-[4-[(4-methyl-1-piperazinyl)methyl]-3-(trifluoromethyl)phenyl]-benzamide.

In another example the dual inhibitor of PDGFRα and FGFR may be lucitanib or a pharmaceutically acceptable salt thereof.

Lucitanib has the CAS registry number 1058137-23-7 and formal name 6-[7-[(1-aminocyclopropyl)methoxy]-6-methoxyquinolin-4-yl]oxy-N-methylnaphthalene-1-carboxamide.

Examples of small molecule inhibitors of PDGFRα include pazopanib (CAS number 444731-52-6), dasatinib (CAS number 302962-49-8), sunitinib (CAS number 557795-19-4). These inhibitors are either approved or currently being evaluated for soft tissue malignancies such as sarcomas and MRTs.

Small molecule inhibitors of FGFR suitable for use in the present invention include NVP-BGJ398 (PubChem CID: 53235510) and AZD4547 (PubChem CID: 51039095) (Tan et al., 2014), TKI258 (dovitinib; PubChem CID: 9886808) and JNJ42756493 (Erdafitinib; PubChem CID: 67462786).

Salts or derivatives of the exemplary inhibitors may be used for the treatment of cancer. As used herein “derivatives” of the therapeutic agents includes salts, coordination complexes, esters such as in vivo hydrolysable esters, free acids or bases, hydrates, prodrugs or lipids, coupling partners.

Salts of the compounds of the invention are preferably physiologically well tolerated and non-toxic. Many examples of salts are known to those skilled in the art. Compounds having acidic groups, such as phosphates or sulfates, can form salts with alkaline or alkaline earth metals such as Na, K, Mg and Ca, and with organic amines such as triethylamine and Tris (2-hydroxyethyl) amine. Salts can be formed between compounds with basic groups, e.g., amines, with inorganic acids such as hydrochloric acid, phosphoric acid or sulfuric acid, or organic acids such as acetic acid, citric acid, benzoic acid, fumaric acid, or tartaric acid. Compounds having both acidic and basic groups can form internal salts.

Esters can be formed between hydroxyl or carboxylic acid groups present in the compound and an appropriate carboxylic acid or alcohol reaction partner, using techniques well known in the art.

Derivatives which as prodrugs of the compounds are convertible in vivo or in vitro into one of the parent compounds. Typically, at least one of the biological activities of compound will be reduced in the prodrug form of the compound, and can be activated by conversion of the prodrug to release the compound or a metabolite of it.

Other derivatives include coupling partners of the compounds in which the compounds is linked to a coupling partner, e.g. by being chemically coupled to the compound or physically associated with it. Examples of coupling partners include a label or reporter molecule, a supporting substrate, a carrier or transport molecule, an effector, a drug, an antibody or an inhibitor. Coupling partners can be covalently linked to compounds of the invention via an appropriate functional group on the compound such as a hydroxyl group, a carboxyl group or an amino group. Other derivatives include formulating the compounds with liposomes.

### Antibodies

Antibodies may be employed in the present invention as an example of a class of inhibitor, and more particularly as inhibitors of PDGFRα and/or FGFR.

Antibodies for use in the invention include the PDGFRα inhibitory antibody Olaratumab. Olaratumab (also IMC-3G3 or LY3012207) selectively binds PDGFRα blocking the binding of its ligand and has the CAS number 1024603-93-7.

Antibodies may also be used in the methods disclosed herein for assessing an individual having cancer, in particular for determining whether the individual has SMARCB1 deficient cancer that might be treatable according to the present invention, or for determining if a cancer expresses FGFR, for example.

An example of an anti-SMARCB1 antibody is purified mouse anti-BAF47 (BD Biosciences, Catalogue number 612110 or 612111), which is used in the examples to determine the presence of SMARCB1 in a tissue sample.

An example of an anti-FGFR1 antibody is rabbit monoclonal antibody ab76464 from abcam [EPR806Y], which binds to human FGFR1, and which is used to determine the presence of FGFR1 in a tissue sample in the examples.

As used herein, the term “antibody” includes an immunoglobulin whether natural or partly or wholly synthetically produced. The term also covers any polypeptide or protein comprising an antibody binding domain. Antibody fragments which comprise an antigen binding domain include Fab, scFv, Fv, dAb, Fd, and diabodies. It is possible to take monoclonal and other antibodies and use techniques of recombinant DNA technology to produce other antibodies or chimeric molecules which retain the specificity of the original antibody. Such techniques may involve introducing DNA encoding the immunoglobulin variable region, or the complementarity determining regions (CDRs), of an antibody to the constant regions, or constant regions plus framework regions, of a different immunoglobulin. See, for instance, EP 0 184 187 A, GB 2,188,638 A or EP 0 239 400 A.

Antibodies can be modified in a number of ways and the term “antibody molecule” should be construed as covering any specific binding member or substance having an antibody antigen-binding domain with the required specificity. Thus, this term covers antibody fragments and derivatives, including any polypeptide comprising an immunoglobulin binding domain, whether natural or wholly or partially synthetic. Chimeric molecules comprising an immunoglobulin binding domain, or equivalent, fused to another polypeptide are therefore included. Cloning and expression of chimeric antibodies are described in EP 0 120 694 A and EP 0 125 023 A.

It has been shown that fragments of a whole antibody can perform the function of binding antigens. Examples of binding fragments are (i) the Fab fragment consisting of VL, VH, CL and CH1 domains; (ii) the Fd fragment consisting of the VH and CH1 domains; (iii) the Fv fragment consisting of the VL and VH domains of a single antibody; (iv) the dAb fragment (Ward, E. S. et al., Nature 341, 544-546 (1989)) which consists of a VH domain; (v) isolated CDR regions; (vi) F(ab′)2 fragments, a bivalent fragment comprising two linked Fab fragments (vii) single chain Fv molecules (scFv), wherein a VH domain and a VL domain are linked by a peptide linker which allows the two domains to associate to form an antigen binding site (Bird et al, Science, 242; 423-426, 1988; Huston et al, PNAS USA, 85: 5879-5883, 1988); (viii) bispecific single chain Fv dimers (WO 93/11161) and (ix) “diabodies”, multivalent or multispecific fragments constructed by gene fusion (WO 94/13804; Holliger et al, P.N.A.S. USA, 90: 6444-6448, 1993); (x) immunoadhesins (WO 98/50431). Fv, scFv or diabody molecules may be stabilised by the incorporation of disulphide bridges linking the VH and VL domains (Reiter et al, Nature Biotech, 14: 1239-1245, 1996). Minibodies comprising a scFv joined to a CH3 domain may also be made (Hu et al, Cancer Res., 56: 3055-3061, 1996).

Preferred antibodies used in accordance with the present invention are isolated, in the sense of being free from contaminants such as antibodies able to bind other polypeptides and/or free of serum components. Monoclonal antibodies are preferred for some purposes, though polyclonal antibodies are within the scope of the present invention.

The reactivities of antibodies on a sample may be determined by any appropriate means. Tagging with individual reporter molecules is one possibility. The reporter molecules may directly or indirectly generate detectable, and preferably measurable, signals. The linkage of reporter molecules may be directly or indirectly, covalently, e.g. via a peptide bond or non-covalently. Linkage via a peptide bond may be as a result of recombinant expression of a gene fusion encoding antibody and reporter molecule. One favoured mode is by covalent linkage of each antibody with an individual fluorochrome, phosphor or laser exciting dye with spectrally isolated absorption or emission characteristics. Suitable fluorochromes include fluorescein, rhodamine, phycoerythrin and Texas Red. Suitable chromogenic dyes include diaminobenzidine.

Other reporters include macromolecular colloidal particles or particulate material such as latex beads that are coloured, magnetic or paramagnetic, and biologically or chemically active agents that can directly or indirectly cause detectable signals to be visually observed, electronically detected or otherwise recorded. These molecules may be enzymes which catalyse reactions that develop or change colours or cause changes in electrical properties, for example. They may be molecularly excitable, such that electronic transitions between energy states result in characteristic spectral absorptions or emissions. They may include chemical entities used in conjunction with biosensors. Biotin/avidin or biotin/streptavidin and alkaline phosphatase detection systems may be employed.

Antibodies according to the present invention may be used in screening for the presence of a polypeptide, for example in a test sample containing cells or cell lysate as discussed, and may be used in purifying and/or isolating a polypeptide according to the present invention, for instance following production of the polypeptide by expression from encoding nucleic acid. Antibodies may modulate the activity of the polypeptide to which they bind and so, if that polypeptide has a deleterious effect in an individual, may be useful in a therapeutic context (which may include prophylaxis).

### Ligand Traps

Another class of inhibitors useful for treating cancer according to the present invention is ligand traps. Ligand traps comprise an antibody regions (e.g. the Fc region) and a ligand binding domain of another protein.

A ligand trap may act as a free form of the target receptor to be inhibited, thus preventing binding of a ligand to the native receptor.

In the context of the present invention, the ligand trap may bind to PDGF or FGF. In other words, the ligand trap may comprise the ligand binding domain of PDGFRα or FGFR, or a variant thereof which binds to PDGF or FGF. For example, the ligand trap may comprise the extracellular domain of FGFR or PDGFRα.

An example of an FGF ligand trap suitable for use in the present invention is FP-1039 (GSK3052230) (Tolcher et al. 2016).

### Peptide Fragments

Another class of inhibitors useful for treating cancer in accordance with the invention is peptide fragments that interfere with the activity of PDGFRα and/or FGFR. Peptide fragments may be generated wholly or partly by chemical synthesis that block the catalytic sites of PDGFRα and/or FGRF. A peptide fragment may interfere with receptor dimerization, for example.

Peptide fragments can be readily prepared according to well-established, standard liquid or, preferably, solid-phase peptide synthesis methods, general descriptions of which are broadly available (see, for example, in J. M. Stewart and J. D. Young, Solid Phase Peptide Synthesis, 2nd edition, Pierce Chemical Company, Rockford, Ill. (1984), in M. Bodanzsky and A. Bodanzsky, The Practice of Peptide Synthesis, Springer Verlag, New York (1984); and Applied Biosystems 430A Users Manual, ABI Inc., Foster City, Calif.), or they may be prepared in solution, by the liquid phase method or by any combination of solid-phase, liquid phase and solution chemistry, e.g. by first completing the respective peptide portion and then, if desired and appropriate, after removal of any protecting groups being present, by introduction of the residue X by reaction of the respective carbonic or sulfonic acid or a reactive derivative thereof.

Other candidate compounds for inhibiting PDGFRα and/or FGFR may be based on modelling the 3-dimensional structure of these receptors and using rational drug design to provide candidate compounds with particular molecular shape, size and charge characteristics. A candidate inhibitor, for example, may be a “functional analogue” of a peptide fragment or other compound which inhibits the component. A functional analogue has the same functional activity as the peptide or other compound in question. Examples of such analogues include chemical compounds which are modelled to resemble the three dimensional structure of the component in an area which contacts another component, and in particular the arrangement of the key amino acid residues as they appear.

### Nucleic Acid Inhibitors

Another class of inhibitors useful for treatment of cancer in accordance with the invention includes nucleic acid inhibitors of PDGFRα and/or FGFR, or the complements thereof, which inhibit activity or function by down-regulating production of active polypeptide. This can be monitored using conventional methods well known in the art, for example by screening using real time PCR.

Expression of FGFR and/or PDGFRα may be inhibited using anti-sense or RNAi technology. The use of these approaches to down-regulate gene expression is now well-established in the art.

Anti-sense oligonucleotides may be designed to hybridise to the complementary sequence of nucleic acid, pre-mRNA or mature mRNA, interfering with the production of the base excision repair pathway component so that its expression is reduced or completely or substantially completely prevented. In addition to targeting coding sequence, anti-sense techniques may be used to target control sequences of a gene, e.g. in the 5′ flanking sequence, whereby the anti-sense oligonucleotides can interfere with expression control sequences. The construction of anti-sense sequences and their use is described for example in Peyman & Ulman, Chemical Reviews, 90:543-584, 1990 and Crooke, Ann. Rev. Pharmacol. Toxicol., 32:329-376, 1992.

Oligonucleotides may be generated in vitro or ex vivo for administration or anti-sense RNA may be generated in vivo within cells in which down-regulation is desired. Thus, double-stranded DNA may be placed under the control of a promoter in a “reverse orientation” such that transcription of the anti-sense strand of the DNA yields RNA which is complementary to normal mRNA transcribed from the sense strand of the target gene. The complementary anti-sense RNA sequence is thought then to bind with mRNA to form a duplex, inhibiting translation of the endogenous mRNA from the target gene into protein. Whether or not this is the actual mode of action is still uncertain. However, it is established fact that the technique works.

The complete sequence corresponding to the coding sequence in reverse orientation need not be used. For example fragments of sufficient length may be used. It is a routine matter for the person skilled in the art to screen fragments of various sizes and from various parts of the coding or flanking sequences of a gene to optimise the level of anti-sense inhibition. It may be advantageous to include the initiating methionine ATG codon, and perhaps one or more nucleotides upstream of the initiating codon. A suitable fragment may have about 14-23 nucleotides, e.g., about 15, 16 or 17 nucleotides.

An alternative to anti-sense is to use a copy of all or part of the target gene inserted in sense, that is the same orientation as the target gene, to achieve reduction in expression of the target gene by co-suppression (Angell & Baulcombe, The EMBO Journal 16(12):3675-3684, 1997 and Voinnet & Baulcombe, Nature, 389: 553, 1997). Double stranded RNA (dsRNA) has been found to be even more effective in gene silencing than both sense or antisense strands alone (Fire et al, Nature 391, 806-811, 1998). dsRNA mediated silencing is gene specific and is often termed RNA interference (RNAi). Methods relating to the use of RNAi to silence genes in C. elegans, Drosophila, plants, and mammals are known in the art (Fire, Trends Genet., 15: 358-363, 19999; Sharp, RNA interference, Genes Dev. 15: 485-490 2001; Hammond et al., Nature Rev. Genet. 2: 110-1119, 2001; Tuschl, Chem. Biochem. 2: 239-245, 2001; Hamilton et al., Science 286: 950-952, 1999; Hammond, et al., Nature 404: 293-296, 2000; Zamore et al., Cell, 101: 25-33, 2000; Bernstein, Nature, 409: 363-366, 2001; Elbashir et al, Genes Dev., 15: 188-200, 2001; WO01/29058; WO99/32619, and Elbashir et al, Nature, 411: 494-498, 2001).

RNA interference is a two-step process. First, dsRNA is cleaved within the cell to yield short interfering RNAs (siRNAs) of about 21-23nt length with 5′ terminal phosphate and 3′ short overhangs (˜2nt). The siRNAs target the corresponding mRNA sequence specifically for destruction (Zamore, Nature Structural Biology, 8, 9, 746-750, 2001.

RNAi may also be efficiently induced using chemically synthesized siRNA duplexes of the same structure with 3′-overhang ends (Zamore et al, Cell, 101: 25-33, 2000). Synthetic siRNA duplexes have been shown to specifically suppress expression of endogenous and heterologeous genes in a wide range of mammalian cell lines (Elbashir et al, Nature, 411: 494-498, 2001).

Another possibility is that nucleic acid is used which on transcription produces a ribozyme, able to cut nucleic acid at a specific site and therefore also useful in influencing gene expression, e.g., see Kashani-Sabet & Scanlon, Cancer Gene Therapy, 2(3): 213-223, 1995 and Mercola & Cohen, Cancer Gene Therapy, 2(1): 47-59, 1995.

Small RNA molecules may be employed to regulate gene expression. These include targeted degradation of mRNAs by small interfering RNAs (siRNAs), post transcriptional gene silencing (PTGs), developmentally regulated sequence-specific translational repression of mRNA by micro-RNAs (miRNAs) and targeted transcriptional gene silencing.

A role for the RNAi machinery and small RNAs in targeting of heterochromatin complexes and epigenetic gene silencing at specific chromosomal loci has also been demonstrated. Double-stranded RNA (dsRNA)-dependent post transcriptional silencing, also known as RNA interference (RNAi), is a phenomenon in which dsRNA complexes can target specific genes of homology for silencing in a short period of time. It acts as a signal to promote degradation of mRNA with sequence identity. A 20-nt siRNA is generally long enough to induce gene-specific silencing, but short enough to evade host response. The decrease in expression of targeted gene products can be extensive with 90% silencing induced by a few molecules of siRNA.

In the art, these RNA sequences are termed “short or small interfering RNAs” (siRNAs) or “microRNAs” (miRNAs) depending on their origin. Both types of sequence may be used to down-regulate gene expression by binding to complimentary RNAs and either triggering mRNA elimination (RNAi) or arresting mRNA translation into protein. siRNA are derived by processing of long double stranded RNAs and when found in nature are typically of exogenous origin. Micro-interfering RNAs (miRNA) are endogenously encoded small non-coding RNAs, derived by processing of short hairpins. Both siRNA and miRNA can inhibit the translation of mRNAs bearing partially complimentary target sequences without RNA cleavage and degrade mRNAs bearing fully complementary sequences.

The siRNA ligands are typically double stranded and, in order to optimise the effectiveness of RNA mediated down-regulation of the function of a target gene, it is preferred that the length of the siRNA molecule is chosen to ensure correct recognition of the siRNA by the RISC complex that mediates the recognition by the siRNA of the mRNA target and so that the siRNA is short enough to reduce a host response.

miRNA ligands are typically single stranded and have regions that are partially complementary enabling the ligands to form a hairpin. miRNAs are RNA genes which are transcribed from DNA, but are not translated into protein. A DNA sequence that codes for a miRNA gene is longer than the miRNA. This DNA sequence includes the miRNA sequence and an approximate reverse complement. When this DNA sequence is transcribed into a single-stranded RNA molecule, the miRNA sequence and its reverse-complement base pair to form a partially double stranded RNA segment. The design of microRNA sequences is discussed in John et al, PLoS Biology, 11(2), 1862-1879, 2004.

Typically, the RNA ligands intended to mimic the effects of siRNA or miRNA have between 10 and 40 ribonucleotides (or synthetic analogues thereof), more preferably between 17 and 30 ribonucleotides, more preferably between 19 and 25 ribonucleotides and most preferably between 21 and 23 ribonucleotides. In some embodiments of the invention employing double-stranded siRNA, the molecule may have symmetric 3′ overhangs, e.g. of one or two (ribo)nucleotides, typically a UU of dTdT 3′ overhang. Based on the disclosure provided herein, the skilled person can readily design suitable siRNA and miRNA sequences, for example using resources such as Ambion's siRNA finder, see http://www.ambion.com/techlib/misc/siRNA_finder.html. siRNA and miRNA sequences can be synthetically produced and added exogenously to cause gene downregulation or produced using expression systems (e.g. vectors). In a preferred embodiment the siRNA is synthesized synthetically.

Longer double stranded RNAs may be processed in the cell to produce siRNAs (e.g. see Myers, Nature Biotechnology, 21: 324-328, 2003). The longer dsRNA molecule may have symmetric 3′ or 5′ overhangs, e.g. of one or two (ribo)nucleotides, or may have blunt ends. The longer dsRNA molecules may be 25 nucleotides or longer. Preferably, the longer dsRNA molecules are between 25 and 30 nucleotides long. More preferably, the longer dsRNA molecules are between 25 and 27 nucleotides long. Most preferably, the longer dsRNA molecules are 27 nucleotides in length. dsRNAs 30 nucleotides or more in length may be expressed using the vector pDECAP (Shinagawa et al., Genes and Dev., 17: 1340-5, 2003).

Another alternative is the expression of a short hairpin RNA molecule (shRNA) in the cell. shRNAs are more stable than synthetic siRNAs. A shRNA consists of short inverted repeats separated by a small loop sequence. One inverted repeat is complimentary to the gene target. In the cell the shRNA is processed by DICER into a siRNA which degrades the target gene mRNA and suppresses expression. In a preferred embodiment the shRNA is produced endogenously (within a cell) by transcription from a vector. shRNAs may be produced within a cell by transfecting the cell with a vector encoding the shRNA sequence under control of a RNA polymerase III promoter such as the human H1 or 7SK promoter or a RNA polymerase II promoter. Alternatively, the shRNA may be synthesised exogenously (in vitro) by transcription from a vector. The shRNA may then be introduced directly into the cell. Preferably, the shRNA sequence is between 40 and 100 bases in length, more preferably between 40 and 70 bases in length. The stem of the hairpin is preferably between 19 and 30 base pairs in length. The stem may contain G-U pairings to stabilise the hairpin structure.

In one embodiment, the siRNA, longer dsRNA or miRNA is produced endogenously (within a cell) by transcription from a vector. The vector may be introduced into the cell in any of the ways known in the art. Optionally, expression of the RNA sequence can be regulated using a tissue specific promoter. In a further embodiment, the siRNA, longer dsRNA or miRNA is produced exogenously (in vitro) by transcription from a vector.

Alternatively, siRNA molecules may be synthesized using standard solid or solution phase synthesis techniques, which are known in the art. Linkages between nucleotides may be phosphodiester bonds or alternatives, e.g., linking groups of the formula P(O)S, (thioate); P(S)S, (dithioate); P(O)NR′2; P(O)R′; P(O)OR6; CO; or CONR′2 wherein R is H (or a salt) or alkyl (1-12C) and R6 is alkyl (1-9C) is joined to adjacent nucleotides through-O-or-S-.

Modified nucleotide bases can be used in addition to the naturally occurring bases, and may confer advantageous properties on siRNA molecules containing them.

For example, modified bases may increase the stability of the siRNA molecule, thereby reducing the amount required for silencing. The provision of modified bases may also provide siRNA molecules, which are more, or less, stable than unmodified siRNA.

The term ‘modified nucleotide base’ encompasses nucleotides with a covalently modified base and/or sugar. For example, modified nucleotides include nucleotides having sugars, which are covalently attached to low molecular weight organic groups other than a hydroxyl group at the 3′position and other than a phosphate group at the 5′position. Thus modified nucleotides may also include 2′substituted sugars such as 2′-O-methyl-; 2-O-alkyl; 2-O-allyl; 2′-S-alkyl; 2′-S-allyl; 2′-fluoro-; 2′-halo or 2; azido-ribose, carbocyclic sugar analogues a-anomeric sugars; epimeric sugars such as arabinose, xyloses or lyxoses, pyranose sugars, furanose sugars and sedoheptulose.

Modified nucleotides are known in the art and include alkylated purines and pyrimidines, acylated purines and pyrimidines, and other heterocycles. These classes of pyrimidines and purines are known in the art and include pseudoisocytosine, N4,N4-ethanocytosine, 8-hydroxy-N6-methyladenine, 4-acetylcytosine,5-(carboxyhydroxylmethyl) uracil, 5 fluorouracil, 5-bromouracil, 5-carboxymethylaminomethyl-2-thiouracil, 5-carboxymethylaminomethyl uracil, dihydrouracil, inosine, N6-isopentyl-adenine, 1-methyladenine, 1-methylpseudouracil, 1-methylguanine, 2,2-dimethylguanine, 2methyladenine, 2-methylguanine, 3-methylcytosine, 5-methylcytosine, N6-methyladenine, 7-methylguanine, 5-methylaminomethyl uracil, 5-methoxy amino methyl-2-thiouracil, -D-mannosylqueosine, 5-methoxycarbonylmethyluracil, 5methoxyuracil, 2 methylthio-N6-isopentenyladenine, uracil-5-oxyacetic acid methyl ester, psueouracil, 2-thiocytosine, 5-methyl-2 thiouracil, 2-thiouracil, 4-thiouracil, 5methyluracil, N-uracil-5-oxyacetic acid methylester, uracil 5-oxyacetic acid, queosine, 2-thiocytosine, 5-propyluracil, 5-propylcytosine, 5-ethyluracil, 5ethylcytosine, 5-butyluracil, 5-pentyluracil, 5-pentylcytosine, and 2,6,diaminopurine, methylpsuedouracil, 1-methylguanine, 1-methylcytosine.

Other inhibitors of FGFR and/or PDGFRα include genome editing systems, for example Clustered Regularly Interspaced Short Palindromic Repeats (CRISPR)/Cas9 systems, zinc finger nucleases (ZFNs), transcription activator-like effector nucleases (TALENs), as well as systems using other nucleases that can cause DNA breaks or bind to DNA. These systems can be used to prevent the expression of functioning FGFR and/or PDGFRα in target cells. Such genome editing systems are also inhibitors within the scope of the present invention.

### Treatment of SMARCB1 Deficient Cancer

In a first aspect the present invention provides methods and medical uses for the treatment of SMARCB1 deficient cancer.

SMARCB1 protein is non-functional if it is not in the nucleus. Accordingly, SMARCB1 deficient cancers are characterised by a lack of SMARCB1 protein in cell nuclei. In other words, SMARCB1 protein is not present in the cell nuclei of SMARCB1 deficient cancer cells.

SMARCB1 deficiency may be caused by a number of mechanisms. In some instances, SMARCB1 may be found in the cell cytoplasm, but not the cell nucleus. SMARCB1 deficiency may be because the SMARCB1 protein itself is not expressed, or because a SMARCB1 mutant is expressed which does not localise to the nucleus, for example. Another reason for SMARCB1 deficiency may be because there is a defect in the mechanism which incorporates it into the SWI/SNF (SWItch/Sucrose Non-Fermentable) complex. By way of example, the SS18-SSX fusion in synovial sarcoma is known to disrupt SWI/SNF assembly resulting in SMARCB1-deficient complexes (Kadoch and Crabtree, 2013).

The uses and methods may comprise the step of determining if the cancer is SMARBC1 deficient. This may involve the step of obtaining a sample from the individual to be treated, and determining the expression of SMARCB1 in a sample obtained from the individual to be treated.

A cancer may be identified as SMARCB1 deficient cancer by carrying out one or more assays or tests on a sample of cells from an individual. The sample will generally be a sample of cancer cells.

SMARBC1 expression may be determined relative to a control, for example in the case of defects in cancer cells, relative to non-cancerous cells, preferably from the same tissue.

By way of example, SMARCB1 expression may be determined by using techniques such as Western blot analysis for SMARCB1 protein, immunohistochemistry, quantitative PCR for the mRNA of SMARCB1, comparative genomic hybridization (e.g. array CGH) for loss of SMARCB1 gene. Examples of such tests in SMARCB1 deficient cancers can be found in Modena et al., 2005.

The determination of SMARCB1 status can be carried out by analysis of SMARCB1 protein expression.

The presence or amount of SMARCB1 protein may be determined using a binding agent capable of specifically binding to the SMARCB1 protein, or fragments thereof. A type of SMARCB1 protein binding agent is an antibody capable of specifically binding the SMARCB1 or fragment thereof. Suitable antibodies include anti-BAF47 available from BD Biosciences (catalog no. 612110).

The antibody may be labelled to enable it to be detected or capable of detection following reaction with one or more further species, for example using a secondary antibody that is labelled or capable of producing a detectable result, e.g. in an ELISA type assay. As an alternative a labelled binding agent may be employed in a western blot to detect SMARCB1 protein.

Preferably, the method for determining the presence of SMARCB1 protein may be carried out on a sample of cancer cells, for example using immunohistochemical (IHC) analysis. IHC analysis can be carried out using paraffin fixed samples or frozen tissue samples, and generally involves staining the samples to highlight the presence and location of SMARCB1 protein.

SMARCB1 deficient tumours can be identified using IHC analysis by the lack of SMARCB1 nuclear staining. Accordingly, in some embodiments the cancer to be treated may have no SMARCB1 protein in the cancer cell nucleus as determined by immunohistochemical analysis.

While some SMARCB1 deficient cancers will show some SMARCB1 staining, it is not localised to the nucleus. Accordingly, SMARCB1 deficient cancers may show no nuclear SMARCB1 staining or no SMARCB1 staining at all, as determined by IHC.

Other methods for determining SMARCB1 status include cytogenetic testing including detection of chromosomal abnormalities, for example by cytogenetic testing. Array CGH (aCGH) may be used to detect 22q deletion indicative of a SMARCB1 deficient cancer. These cancers may have a structural rearrangement at 22q, in particular a focal deletion in 22q11.23.

Alternatively or additionally, the determination of SMARCB1 gene expression may involve determining the presence or amount of SMARCB1 mRNA in a sample. Methods for doing this are well known to the skilled person. By way of example, they include determining the presence of SMARCB1 mRNA; and/or (ii) using PCR involving one or more primers based on a SMARCB1 nucleic acid sequence to determine whether the SMARCB1 transcript is present in a sample. The probe may also be immobilised as a sequence included in a SMARCB1.

Detecting SMARCB1 mRNA may carried out by extracting RNA from a sample of the tumour and measuring SMARCB1 expression specifically using quantitative real time RT-PCR. Alternatively or additionally, the expression of SMARCB1 could be assessed using RNA extracted from a sample of cancer cells for an individual using microarray analysis, which measures the levels of mRNA for a group of genes using a plurality of probes immobilised on a substrate to form the array.

A number of cancer types harbour SMARCB1 deficiencies including cribiform neuroepithelial tumour of the ventricle, epithelioid sarcomas, renal medullary carcinoma, epithelioid malignant peripheral nerve sheath tumours and extraskeletal myxoid chondrosarcomas. A subset of collecting duct carcinomas are also SMARCB1 deficient. The SS18-SSX fusion in synovial sarcoma is known to disrupt SWI/SNF assembly resulting in SMARCB1-deficient complexes (Kadoch and Crabtree, 2013). Furthermore, reduced SMARCB1 protein expression is found in a proportion of synovial sarcomas (Kohashi et al. 2010, Rekhi et al. 2015).

Accordingly, the cancer to be treated according to the present invention may be selected from rhabdoid tumours including malignant rhabdoid tumours (MRT) and atypical teratoid rhabdoid tumours (AT/RT), epithelioid sarcoma, renal medullary carcinoma, epithelioid malignant peripheral nerve sheath tumour, extraskeletal myxoid chondrosarcoma, cribiform neuroepithelial tumour of the ventricle, collecting duct carcinoma and synovial sarcomas. The cancer to be treated may be a rhabdoid tumour, for example MRT.

The rhabdoid tumour may be in the kidney, liver, soft tissue or central nervous system, e.g. intracerebral. The rhabdoid tumour may be in the kidney or may be intracerebral.

The individual to be treated is preferably a mammal, in particular a human. SMARCB1 deficient cancers to be treated according to the present invention (especially MRTs) may be paediatric cancers. In other words, the individual to be treated may be a child. In some embodiments, the cancer is a paediatric MRT. The individual may be less than 18, 15, 10, 5, 3, or 2 years of age. For example, the individual may be less than 2 years of age.

Some SMARCB1 deficient cancers are more common in adults, for example epithelioid sarcomas. Accordingly, in some embodiments the individual to be treated is an adult.

In some embodiments the cancer to be treated is resistant to treatment with a PDGFRα inhibitor alone.

Resistance to a PDGFRα inhibitor can be determined by monitoring of tumour size and metastasis over the course of treatment with a PDGFRα inhibitor.

Tumour size and metastasis can be determined by imaging the individual. Suitable imaging methods are known to the skilled person, such as CT scans and MRI scans.

The individual to be treated may be imaged regularly and the size of the tumour measured. Tumour growth (increase in tumour size) indicates that the tumour is resistant to the PDGFRα inhibitor. Similarly metastasis indicates that the tumour is resistant to the PDGFα inhibitor. Shrinking or stable tumour size would indicate that the tumour is not resistant to the PDGFRα inhibitor. In some embodiments, resistance may be indicated by initial shrinking or stabilisation of tumour size, followed by increase in tumour size or metastasis over the course of treatment with a PDGFRα inhibitor alone.

The individual may be imaged at regular intervals over the course of PDGFRα inhibitor treatment. For example, the individual may be imaged every 1-16 weeks, 2-12 weeks or 4-10 weeks. For example the individual may be imaged every 4-10 weeks.

Thus in some embodiments the methods of treatment comprise selecting an individual for treatment with an FGFR inhibitor where the tumour has grown and/or metastasized after treatment with a PDGFRα inhibitor.

For example the individual being treated with a PDGFRα inhibitor may be imaged multiple times to monitor tumour size and metastasis. Where the tumour grows and/or further metastasizes after treatment with the PDGFRα inhibitor, the individual is treated with a FGFR inhibitor (e.g. an FGFR1 inhibitor).

For example, in the methods and uses of an FGFR inhibitor for the treatment of a SMARCB1 deficient cancer that is resistant to treatment with a PDGFRα inhibitor alone, resistance to a PDGFRα inhibitor is determined by tumour growth and/or metastasis after treatment with a PDGFRα inhibitor alone.

Cancers which are resistant to PDGFRα inhibitors may also have altered expression of PDGFRα, such as increased or decreased expression. In some embodiments, the PDGFRα inhibitor resistant tumours may have reduced expression of PDGFRα relative to SMARCB1 deficient cancer cells that are not resistant, or loss of PDGFRα expression, for example. In other embodiments, PDGFRα expression is upregulated in resistant cells. In some embodiments of the methods and uses, the individual to be treated may be tested for loss of PDGFRα expression or reduced expression of PDGFRα. The methods and uses may comprise testing a sample of cancer cells for loss of PDGFRα expression or reduced expression of PDGFRα.

Generally, MRT have elevated expression levels of both PDGFRα and FGFR, e.g. FGFR1. Accordingly, the cancer to be treated by have elevated expression levels of one or both of PDGFRα and FGFR, as compared to a normal tissue sample. In some embodiments of the methods and uses, the individual to be treated may be tested for increased expression of FGFR, e.g. FGFR1, and/or PDGFRα. The methods and uses may comprise testing a tumour sample (a sample of tumour cells) for increased expression of FGFR and/or PDGFRα.

Expression can be determined in tissue samples using standard techniques. For example, gene expression can be determined by measuring mRNA levels, e.g. using real-time quantitative PCR.

Preferably, IHC is used to detect protein expression, in a sample. Suitable antibodies for this purpose are disclosed in the examples. FGFR and/or PDGFRα may show increased cytoplasmic or membrane staining in cancers to be treated. Any of the methods described above in relation to determining SMARCB1 expression may be used to determine expression of PDGFRα or FGFR, e.g. FGFR1.

### Treatment of Pazopanib Resistant Cancer

In a second aspect, the invention provides methods and medical uses for the treatment of pazopanib resistant cancer. The uses and methods may involve treatment of a cancer which has been determined to be resistant to pazopanib. The uses and methods may comprise the step of determining if the cancer is resistant to pazopanib. Resistance to pazopanib can be determined by monitoring the tumour size and metastasis over the course of treatment with pazopanib.

Tumour size and metastasis can be determined by imaging the individual. Suitable imaging methods are known to the skilled person, such as CT (computerized tomography) scans and MRI (magnetic resonance imaging) scans.

The individual to be treated may be imaged regularly and the size of the tumour measured. Tumour growth (increase in tumour size) indicates that the tumour is resistant to pazopanib treatment. Similarly metastasis indicates that the tumour is resistant to pazopanib treatment. Shrinking or stable tumour size would indicate that the tumour is not resistant to pazopanib. In some embodiments, resistance may be indicated by initial shrinking or stabilisation of tumour size, followed by increase in tumour size or metastasis over the course of treatment with pazopanib.

The individual may be imaged at regular intervals over the course of pazopanib treatment. For example, the individual may be imaged every 1-16 weeks, 2-12 weeks or 4-10 weeks. For example the individual may be imaged every 4-10 weeks. The individual may be imaged before the start of treatment and over the course of the treatment.

Thus in some embodiments the methods of treatment comprise selecting an individual for treatment with an FGFR inhibitor where the tumour has grown and/or metastasized after treatment with pazopanib.

For example the individual being treated with pazopanib may be imaged multiple times to monitor tumour size and metastasis. Where the tumour grows and/or further metastasizes after treatment with pazopanib, the individual is treated with a FGFR inhibitor (e.g. an FGFR1 inhibitor).

For example, in the methods and uses of an FGFR inhibitor for the treatment of a pazopanib resistant cancer, resistance to pazopanib is determined by tumour growth and/or metastasis after treatment with pazopanib.

In some embodiments the methods comprise the steps of treating a cancer in an individual with pazopanib, and when the cancer becomes pazopanib resistant, then treating the cancer with an FGFR inhibitor. As above, determination of pazopanib resistance may be indicated by tumour growth and/or metastasis after treatment with pazopanib. The tumour growth and/or presence of metastasis may be monitored using conventional imaging techniques.

In some embodiments, the methods further comprise the step of determining FGFR expression (e.g. protein expression) in the cancer. For example determining FGFR1 expression. For example, a sample of cancer cells may be obtained from the individual, and tested for expression of FGFR.

Thus, the inhibitors of FGFR may be used in the treatment of a pazopanib resistant cancer in an individual, where the cancer expresses FGFR (e.g. FGFR1).

Expression can be determined in tissue samples using standard techniques. For example, gene expression can be determined by measuring mRNA levels, e.g. using real-time quantitative PCR. Preferably, IHC is used to detect protein expression, in a sample. Suitable antibodies for this purpose are disclosed in the examples. FGFR may show increased cytoplasmic or membrane staining in cancers to be treated.

Any of the methods described elsewhere herein in relation to determining SMARCB1 expression may be used to determine expression of FGFR, e.g. FGFR1. By way of example, the presence or amount of FGFR protein may be determined using a binding agent capable of specifically binding to FGFR protein, or fragments thereof. A type of FGFR protein binding FGFR is an antibody capable of specifically binding FGFR or a fragment thereof. Suitable antibodies include anti-FGFR1 available from abcam (product code ab76464).

The binding agent (e.g. antibody) may be labelled to enable it to be detected or be capable of detection following reaction with one or more further species, for example using a secondary antibody that is labelled or capable of producing a detectable result, e.g. in an ELISA type assay. As an alternative a labelled binding agent may be employed in a western blot to detect FGFR protein.

Preferably, the method for determining the presence of FGFR protein may be carried out on a sample of cancer cells, for example using immunohistochemical (IHC) analysis. IHC analysis can be carried out using paraffin fixed samples or frozen tissue samples, and generally involves staining the samples to highlight the presence and location of FGFR protein.

Alternatively or additionally, the determination of FGFR gene expression may involve determining the presence or amount of FGFR mRNA in a sample. Methods for doing this are well known to the skilled person. By way of example, they include determining the presence of FGFR mRNA; and/or (ii) using PCR involving one or more primers based on a FGFR nucleic acid sequence to determine whether the FGFR transcript is present in a sample. The probe may also be immobilised as a sequence included in a FGFR.

Detecting FGFR mRNA may carried out by extracting RNA from a sample of the tumour and measuring FGFR expression specifically using quantitative real time RT-PCR. Alternatively or additionally, the expression of FGFR could be assessed using RNA extracted from a sample of cancer cells using microarray analysis, which measures the levels of mRNA for a group of genes using a plurality of probes immobilised on a substrate to form the array.

A number of cancers can be treated with pazopanib. The pazopanib resistant cancer may be a soft tissue sarcoma or renal cell carcinoma. Examples include synovial sarcoma, leiomyosarcoma and solitary fibrous tumours.

The individual to be treated is preferably a mammal, in particular a human.

### Administration and Pharmaceutical Compositions

The active agents disclosed herein for the treatment of SMARCB1 deficient cancer, such as MRT, according to the first aspect of the invention, or for the treatment of pazopanib resistant cancers according to the second aspect of the invention, may be administered alone, but it is generally preferable to provide them in pharmaceutical compositions that additionally comprise with one or more pharmaceutically acceptable carriers, adjuvants, excipients, diluents, fillers, buffers, stabilisers, preservatives, lubricants, or other materials well known to those skilled in the art and optionally other therapeutic or prophylactic agents. Examples of components of pharmaceutical compositions are provided in Remington's Pharmaceutical Sciences, 20th Edition, 2000, pub. Lippincott, Williams & Wilkins.

The term “pharmaceutically acceptable” as used herein includes compounds, materials, compositions, and/or dosage forms which are, within the scope of sound medical judgement, suitable for use in contact with the tissues of a subject (e.g. human) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation.

The active agents disclosed herein for the treatment of SMARCB1 deficient cancer or pazopanib resistant cancer are preferably for administration to an individual in a “prophylactically effective amount” or a “therapeutically effective amount” (as the case may be, although prophylaxis may be considered therapy), this being sufficient to show benefit to the individual. For example, the agents (inhibitors) may be administered in amount sufficient to delay tumour progression, or prevent tumour growth and/or metastasis or to shrink tumours. For example, the agents may be administered in an amount sufficient to induce apoptosis of cancer cells.

The actual amount administered, and rate and time-course of administration, will depend on the nature and severity of what is being treated. Prescription of treatment, e.g. decisions on dosage etc., is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 20th Edition, 2000, Lippincott, Williams & Wilkins. A composition may be administered alone or in combination with other treatments, either simultaneously or sequentially, dependent upon the condition to be treated.

The formulations may conveniently be presented in unit dosage form and may be prepared by any methods well known in the art of pharmacy. Such methods include the step of bringing the active compound into association with a carrier, which may constitute one or more accessory ingredients. In general, the formulations are prepared by uniformly and intimately bringing into association the active compound with liquid carriers or finely divided solid carriers or both, and then if necessary shaping the product.

The agents disclosed herein for the treatment of SMARCB1 deficient cancer or pazopanib resistant cancer may be administered to a subject by any convenient route of administration, whether systemically/peripherally or at the site of desired action, including but not limited to, oral (e.g. by ingestion); topical (including e.g. transdermal, intranasal, ocular, buccal, and sublingual); pulmonary (e.g. by inhalation or insufflation therapy using, e.g. an aerosol, e.g. through mouth or nose); rectal; vaginal; parenteral, for example, by injection, including subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal; by implant of a depot, for example, subcutaneously or intramuscularly.

Formulations suitable for oral administration (e.g., by ingestion) may be presented as discrete units such as capsules, cachets or tablets, each containing a predetermined amount of the active compound; as a powder or granules; as a solution or suspension in an aqueous or non-aqueous liquid; or as an oil-in-water liquid emulsion or a water-in-oil liquid emulsion; as a bolus; as an electuary; or as a paste.

Formulations suitable for parenteral administration (e.g., by injection, including cutaneous, subcutaneous, intramuscular, intravenous and intradermal), include aqueous and non-aqueous isotonic, pyrogen-free, sterile injection solutions which may contain anti-oxidants, buffers, preservatives, stabilisers, bacteriostats, and solutes which render the formulation isotonic with the blood of the intended recipient; and aqueous and non-aqueous sterile suspensions which may include suspending agents and thickening agents, and liposomes or other microparticulate systems which are designed to target the compound to blood components or one or more organs. Examples of suitable isotonic vehicles for use in such formulations include Sodium Chloride Injection, Ringer's Solution, or Lactated Ringer's Injection. Typically, the concentration of the active compound in the solution is from about 1 ng/ml to about 10 μg/ml, for example from about 10 ng/ml to about 1 μg/ml. The formulations may be presented in unit-dose or multi-dose sealed containers, for example, ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules, and tablets.

Formulations may be in the form of liposomes or other microparticulate systems which are designed to target the active compound to blood components or one or more organs.

Compositions comprising agents disclosed herein for the treatment SMARCB1 deficient cancer or pazopanib resistant cancer may be used in the methods described herein in combination with standard chemotherapeutic regimes or in conjunction with radiotherapy. Examples of other chemotherapeutic agents include Amsacrine (Amsidine), Bleomycin, Busulfan, Capecitabine (Xeloda), Carboplatin, Carmustine (BCNU), Chlorambucil(Leukeran), Cisplatin, Cladribine(Leustat), Clofarabine (Evoltra), Crisantaspase (Erwinase), Cyclophosphamide, Cytarabine (ARA-C), Dacarbazine (DTIC), Dactinomycin (Actinomycin D), Daunorubicin, Docetaxel (Taxotere), Doxorubicin, Epirubicin, Etoposide (Vepesid, VP-16), Fludarabine (Fludara), Fluorouracil (5-FU), Gemcitabine (Gemzar), Hydroxyurea (Hydroxycarbamide, Hydrea), Idarubicin (Zavedos). Ifosfamide (Mitoxana), Irinotecan (CPT-11, Campto), Leucovorin (folinic acid), Liposomal doxorubicin (Caelyx, Myocet), Liposomal daunorubicin (DaunoXome®) Lomustine, Melphalan, Mercaptopurine, Mesna, Methotrexate, Mitomycin, Mitoxantrone, Oxaliplatin (Eloxatin), Paclitaxel (Taxol), Pemetrexed (Alimta), Pentostatin (Nipent), Procarbazine, Raltitrexed (Tomudex®), Streptozocin (Zanosar®), Tegafur-uracil (Uftoral), Temozolomide (Temodal), Teniposide (Vumon), Thiotepa, Tioguanine (6-TG) (Lanvis), Topotecan (Hycamtin), Treosulfan, Vinblastine (Velbe), Vincristine (Oncovin), Vindesine (Eldisine) and Vinorelbine (Navelbine).

Methods of determining the most effective means and dosage of administration are well known to those of skill in the art and will vary with the formulation used for therapy, the purpose of the therapy, the target cell being treated, and the subject being treated. Single or multiple administrations can be carried out with the dose level and pattern being selected by the treating physician.

In general, a suitable dose of the active compound is in the range of about 100 μg to about 250 mg per kilogram body weight of the subject per day. Where the active compound is a salt, an ester, prodrug, or the like, the amount administered is calculated on the basis of the parent compound, and so the actual weight to be used is increased proportionately.

In the context of SMARCB1 deficient cancer, the methods and treatments of the invention may be referred to as a combination therapy or combined treatment. For example, the PDGFRα inhibitor may be used in combination with the FGFR inhibitor. Their use “in combination” denotes any form of concurrent or parallel treatment with a PDGFRα inhibitor and a FGFR inhibitor, and includes the use of a single dual PDGFRα and FGFR inhibitor.

Administration of the PDGFRα inhibitor and the FGFR inhibitor may be in the same composition or in separate compositions. In one aspect a pharmaceutical composition comprising PDGFRα inhibitor and the FGFR inhibitor is provided, where PDGFRα inhibitor and the FGFR inhibitor are different.

Where the PDGFRα inhibitor and the FGFR inhibitor are in the same composition, administration of the two tyrosine kinase inhibitors is simultaneous.

In other embodiments, the PDGFRα inhibitor and the FGFR inhibitor are in separate compositions and may be administered simultaneously or sequentially. Sequential administration means that the PDGFRα inhibitor is administered prior to or after administration of the FGFR inhibitor.

The administration period of the PDGFRα inhibitor and the FGFR inhibitor may overlap. Alternatively, the administration period of the PDGFRα inhibitor and the administration of the FGFR inhibitor do not overlap.

Where the inhibitors are not administered at the same time, they should be administered sufficiently close in time to for the synergistic effect against the cancer cells to occur and/or to induce apoptosis of the cancer cells, and/or for the cancer cells to be sensitized to the PDGFRα inhibitor or to prevent the cells from acquiring resistance to the PDGFRα inhibitor.

In the case of SMARCB1 deficient cancers, the inhibitors of FGFR and/or PDGFRα may be administered in an amount which is effective for achieving a synergistic effect against the cancer cells. The inhibitors may be administered in an amount which induces apoptosis of the cancer cells. The FGFR inhibitor may be present in an amount sufficient to sensitize the cancer cells to a PDGFRα inhibitor or prevent the cells from acquiring resistance to a PDGFRα inhibitor or delay onset of acquired resistance to a PDGFRα inhibitor.

### Medical Uses

In the first aspect disclosed herein, the present invention relates to the treatment of SMARCB1 deficient cancer by dual inhibition PDGFRα and FGFR.

The invention covers an inhibitor of PDGFRα and an inhibitor of FGFR for use in a method of treating a SMARCB1 deficient cancer. In other words a receptor tyrosine kinase inhibitor is provided for use in a method of treating a SMARCB1 deficient cancer, the method comprising inhibition of the receptor tyrosine kinases PDGFRα and FGFR.

The treatments disclosed may be described including the step of administering the active ingredient(s) (the inhibitor(s)) to the individual, e.g. in a therapeutically effective amount.

These treatments may also be described in other wording, for example as below:

One or more receptor tyrosine kinase inhibitors for use in a method of treating a SMARCB1 deficient cancer, wherein the receptor tyrosine kinase inhibitor(s) collectively inhibit PDGFRα and FGFR.

A combination of an inhibitor of PDGFRα and an inhibitor of FGFR for use in a method of treating a SMARCB1 deficient cancer.

An inhibitor of PDGFRα for use in a method of treating SMARCB1 deficient cancer, the method comprising administration of an inhibitor of FGFR.

An inhibitor of FGFR for use in a method of treating a SMARCB1 deficient cancer, the method comprising administration of an inhibitor of PDGFRα.

A composition comprising an inhibitor of PDGFRα and an inhibitor of FGFR1 for use in a method of treating a SMARCB1 deficient cancer.

Use of an inhibitor of PDGFRα and an inhibitor of FGFR in the manufacture of a medicament for treating a SMARCB1 deficient cancer.

A method of treating a SMARCB1 deficient cancer in an individual comprising administration of a receptor tyrosine kinase inhibitor to inhibit PDGFRα and FGFR.

Also provided is an FGFR inhibitor for use in a method of sensitizing cancer cells to a PDGFRα inhibitor in the treatment of cancer, the method comprising administering an FGFR1 inhibitor and a PDGFRα inhibitor. The FGFR inhibitor may also be described as for use in decreasing drug resistance or preventing drug resistance to a PDGFRα inhibitor.

Also provided is an FGFR inhibitor for use in a method of treatment of a SMARCB1 deficient cancer in an individual, where the SMARCB1 deficient cancer is resistant to treatment with a PDGFRα inhibitor, the method comprising administering the FGFR inhibitor to the individual.

The methods and treatments disclosed herein may involve the steps of determining whether a patient is suitable for treatment.

The methods may involve the step of determining that the cancer is SMARCB1 deficient, and selecting such patients for treatment.

For example, the methods may involve the steps of:
a) obtaining a sample of cancer cells from an individual
b) determining SMARCB1 expression in the cancer cells
c) administering an inhibitor of PDGFRα and an inhibitor of FGFR if the cancer is SMARCB1 deficient.

The determining step may involve determination of the presence of absence of SMARCB1 protein in the cancer cell nuclei, where an absence of SMARCB1 protein in the cell nucleus means the cancer is SMARCB1 deficient. The determining step may be carried out using IHC for example.

The methods may involve determining that FGFR is overexpressed as compared to normal tissue expression levels. For example, determining if FGFR1 is over expressed. Patients where FGFR is overexpressed may be selected for the combined treatment of the present invention.

In the second aspect, the invention relates to the use of an FGFR inhibitor for the treatment of pazopanib resistant cancer.

The treatments disclosed may be described including the step of administering the active ingredient(s) (the FGFR inhibitor) to the individual, e.g. in a therapeutically effective amount.

These treatments may also be described in other wording, for example as below:

A composition comprising an inhibitor of FGFR for use in a method of treating a pazopanib resistant cancer.

Use of an inhibitor of FGFR in the manufacture of a medicament for treating a pazopanib resistant cancer.

A method of treating a pazopanib resistant cancer in an individual comprising administration of an FGFR inhibitor to the individual.

The methods and treatments disclosed herein may involve the steps of determining whether a patient is suitable for treatment. The methods may involve the step of determining that the cancer is pazopanib resistant, and selecting such patients for treatment.

For example, the methods may involve the steps of:
a) treating the individual with pazopanib
b) determining whether the cancer is resistant to pazopanib
c) selecting the individual for treatment with if cancer is resistant to treatment with pazopanib
d) treating the individual with an FGFR inhibitor.

For example, the methods may involve the steps of:
a) treating the individual with pazopanib
b) determining tumour size and/or the presence of metastasis in the individual
c) selecting the individual for treatment with if the tumour size increases or metastasises after treatment with pazopanib
d) treating the individual with an FGFR inhibitor.

Tumour size and/or metastasis may be monitored over the course of pazopanib treatment. The individual may be imaged to determine tumour size and/or the presence of metastasis.

In some embodiments the cancer may be selected if it also expresses FGFR, for example FGFR protein. For example, the methods may involve the steps of:
) obtaining a sample of cancer cells from an individual
ii) determining FGFR (e.g. FGFR1) expression in the cancer cells
iii) administering an inhibitor of FGFR if the cancer expresses FGFR.

The determining step may be carried out using IHC for example. These steps may be carried out after the cancer is determined to be pazopanib resistant.

Accordingly the individual to be treated may have been determined to have pazopanib resistant cancer, optionally which expresses FGFR, prior to treatment.

### Other Inhibitor Combinations

Although the description in relation to the treatment of SMARCB1 deficient cancer is directed primarily toward the inhibition of both PDGFRα and FGFR, it is also envisaged that other inhibitor combinations could be effective at treating SMARCB1 deficient cancers, in particular in overcoming resistance to PDGFRα inhibitors alone. Similarly other combinations of inhibitors may be used to treat pazopanib resistant cancers.

The inventors present the first mechanism of acquired resistance to pazopanib in soft tissue malignancies through PDGFRα loss. Phosphoproteomic analysis of the Pazopanib resistant cells revealed candidate target pathways such as PLCG1 and Src family kinases (YES1, FYN and FGR) which are upregulated.

Accordingly, in the first aspect of the invention uses, methods of treatment and compositions as described herein can comprise a combination of a PDFRα inhibitor and an inhibitor of PLCG1, YES1, FYN or FGR. In other words, the FGFR inhibitor may be replaced with an inhibitor of any of PLCG1, YES1, FYN and FGR. These inhibitors can be used to sensitize cancer cells which are resistant to treatment with a PDGFRα inhibitor alone.

In some embodiments an inhibitor of PLCG1, YES1, FYN and/or FGR may be used in combination with the PDGFRα inhibitor and FGFR inhibitor in the methods, uses and compositions of the invention.

In the second aspect of the invention uses, methods of treatment and compositions as described herein can comprise the use of an inhibitor of PLCG1, YES1, FYN or FGR. In other words, the FGFR inhibitor may be replaced with an inhibitor of any of PLCG1, YES1, FYN and FGR. These inhibitors can be used to treat pazopanib resistant cancer cells.

In some embodiments an inhibitor of PLCG1, YES1, FYN and/or FGR may be used in combination with the FGFR inhibitor in the methods, uses and compositions of the invention.

### EXAMPLES

### Experimental Procedures

### Cell Culture

A204 and G402 cells were obtained from ATCC.

### Cell Culture and Derivation of Acquired Resistant Sublines

Cells were cultured in DMEM (A204, G402, Saos2, U2OS, HT1080, SW684, SW872, SW982, Hs729T, RUCH-3, T9195 and AN3CA), RPMI (RMS-YM and SJSA-1) or McCoy5A (MES-SA) media supplemented with 10% FBS/2 mM glutamine/100 units/ml penicillin/100 mg/ml streptomycin in 95% air/5% CO₂ atmosphere at 37° C. For SILAC experiments, A204 cells and resistant sublines were cultured in SILAC DMEM media (Thermo Fisher Scientific) supplemented with light lysine and arginine (ROKO) (Sigma) and heavy lysine and arginine (R10K8) (Goss Scientific) respectively.

Dasatinib, Pazopanib and Sunitinib (LC laboratories) were used to induce resistance in the A204 cells. Cells were grown initially in DMEM media containing drug concentration of 500 nM. The drug was incremented when the cells had proliferated to near confluency alongside minimal visible cell death. Drug concentration was incremented from 2 μM, 3 μM and 5 μM in a stepwise manner over 6 weeks. A final drug concentration of 5 μM was maintained in resistant cells. Media and drug were replenished twice weekly.

### Molecular Biology and Lentiviral Infection

Procedure for ectopic expression of SMARCB1 by lentiviral infection. The pCDH-EF1-PURO-SMARCB1 plasmid was produced by PCR amplifying the whole SMARCB1 coding sequence from pCDNA 3.1-SMARCB1 (a gift from Frederique Quignon, Institute Curie). Restriction sites for XbaI and BamH1 were added to the Forward and Reverse primers respectively. The PCR product was digested and directionally ligated into the multiple cloning site of pCDH-EF1-Puro (Systems Biosciences).

PCDH-CMV-MCS-EF1-SMARCB1 Puro plasmid (System Bioscences) was transiently transfected into HEK293T cells using Calcium Phosphate Transfection method (CalPhos Transfection Kits, Clontech) according to manufacturer's instructions. Lentiviral infection of rhabdoid cells was carried out aiming to transduce about 60%-80% of the total amount of cells in each experiment, using an MOI of 10. To select for infected cells, Puromycin (Invitrogen) was added to the media to a final concentration of 1 μg/mL for 72 hours prior to cell lysis.

### Immunoblotting, Immunoprecipitation and Immunofluorescence

After the indicated treatments, cells were lysed in RIPA lysis buffer at 4° C. Lysates were loaded onto SDS-PAGE gels followed by blotting onto PVDF membranes.

Details of antibodies, immunoprecipitation and immunofluorescence analyses are as follows. For immunoblotting, cells were lysed in RIPA lysis buffer supplemented with protease and phosphatase inhibitors (Thermo Pierce) at 4° C. Lysates were loaded onto SDS-PAGE gels followed by blotting onto PVDF membranes as described (Iwai et al., 2013). Blots were probed with primary antibodies followed by corresponding horseradish peroxidase-conjugated secondary antibodies. Primary antibodies include anti-PDGFRα #3174, CST; anti-pAKT (S473) #4058, CST; anti-AKT #4691, CST; anti-pERK-T202/Y204 #4370, CST; anti-ERK #9102, CST; anti-FGFR1 #76464, abcam; anti-BAF47 (SMARCB1) #61211, BD; anti-TFR #13-6890, ThermoFisherScientific; anti-pY1000 #8954, CST and anti-α-Tubulin #T5168, Sigma. Secondary antibodies include Polyclonal Goat Anti-Rabbit HRP #P0448, Dako and Anti-Mouse HRP #G32-62G-1000, Signalchem. Immunoreactive bands were visualized by chemiluminescence (Amersham) and the blots were exposed to x-ray XAR film (Kodak).

For immunoprecipitation, cells were lysed in RIPA lysis buffer (contained 1% Triton) supplemented with protease and phosphatase inhibitors (Thermo Pierce) at 4° C. After microcentrifugation at 2,000 rpm. for 10 min, 200 μg of lysate was diluted in 200 ul lysis buffer. Primary antibody (anti-PDGFR #3174, CST) was added at 1 mg/ml and incubate with rotation overnight at 4° C. Protein G plus agarose beads were added and incubated for three hours at 4° C. to collect immune complexes, washed five times with lysis buffer and eluted in sample buffer. Proteins were resolved by SDS-PAGE, transferred to PVDF membrane and immunoblotting was performed as described above.

For immunofluorescence experiments, cells were fixed with 4% formaldehyde for 15 min, permeabilised with 0.2% Triton-X 100/PBS for 10 min and then blocked with IF buffer (3% BSA, 0.05% Tween 20 in PBS) for 1h. Specimens were incubated overnight with primary antibodies (anti-PDGFR #3174, CST; anti-FGFR1 # PA5-18344, Thermo Fisher Scientific) at 4° C. rinsed three times with IF buffer and then incubated with secondary antibodies (anti-rabbit Alexa488 and anti-goat Alexa555, Thermo Fisher Scientific). DNA was visualised by DAPI staining. Images were captured using a Zeiss 710 Confocal Microscope.

### Cell Viability and Apoptosis Assays

2000 cells/well were seeded in a 96-well plate and treated with inhibitors at the indicated dose and combinations for 24 h for apoptosis measurement by Caspase-Glo 3/7 Assay (Promega), or for 72 hours in cell viability measurements by WST-1 (Abcam), following the manufacturer's recommendations. IC₅₀ data were generated from dose-response curves fitted using a four-parameter regression fit in PRISM 5 software (GraphPad).

Details for annexin V staining and siRNA transfections are as follows. For Annexin V staining, 3000 cells/well were seeded into 96-well CellCarrier plates (Perkin Elmer). 24 h after seeding, drugs were added and incubated for an additional 48 h. FITC-Annexin V (BD Biosciences) and Hoechst 33342 (Tocris) diluted in 10× annexin binding buffer (0.1M HEPES, 1.4M NaCl, 25 mM CaCl₂) was added and incubated at 37° C. for 15 minutes. Plates were imaged using an Operetta high-content imager (Perkin Elmer). Images were analysed using Harmony software (Perkin Elmer), and annexin positivity defined as number of annexin-FITC-positive cells relative to total number of Hoechst-positive nuclei. The interaction between drugs was analysed by the Chou and Talalay median effect principle as described (Todd et al., 2014). siRNA transfections were performed as follows, 2000 cells/well were reverse transfected in 96-well plates with SMARTpool siRNAs (Dharmacon) using Lullaby reagent (Oz Biosciences). Where indicated, cells were treated with vehicle or drug 24 h post transfection. Apoptosis and cell viability were measured using Caspase 3/7 Glo and Cell Titre Glo (Promega), respectively, 72-96 h post transfection according to manufacturer's instructions and normalised to cells transfected with a non-targeting siRNA pool.

### aCGH, Gene Expression and Phosphoproteomic Analysis

For aCGH analysis, genomic DNA was extracted as previously described (Marchio et al., 2008; Natrajan et al., 2009). The aCGH platform was constructed in-house and comprises ˜32,000 BAC clones tiled across the genome. This platform has been shown to be as robust as, and to have comparable resolution with, high-density oligonucleotide arrays (Coe et al., 2007; Gunnarsson et al., 2008). aCGH data were pre-processed and analyzed using the Base.R script in R version 2.14.0, as previously described (Natrajan et al., 2014). Genomic DNA from each sample was hybridized against a pool of normal female DNA derived from peripheral blood. Raw Log₂ ratios of intensity between samples and pooled female genomic DNA were read without background subtraction and normalized in the LIMMA package in R using PrinTipLoess. Outliers were removed based upon their deviation from neighboring genomic probes, using an estimation of the genome-wide median absolute deviation of all probes. Log₂ ratios were rescaled using the genome wide median absolute deviation in each sample and then smoothed using circular binary segmentation (cbs) in the DNACopy package as described (Natrajan et al., 2009). After filtering polymorphic BACs and BACs mapping to chromosome Y, a final dataset of 31,157 clones with unambiguous mapping information according to build hg19 of the human genome (http://www.ensembl.org). A categorical analysis was applied to the BACs after classifying them as representing amplification (>0.45), gain (>0.08 and ≤0.45), loss (<-0.08) or no change, according to their cbs-smoothed log2 ratio values (Marchio et al., 2008; Natrajan et al., 2009). Threshold values were determined and validated as previously described (Natrajan et al., 2009).

RNA was extracted and gene expression analysis performed on Illumina HTv12 chip as per manufacturer's recommendations. The Illumina Bead Chip (HumanHG-12 v4) data were pre-processed, log2-transformed, and quantile normalized using the beadarray package in Bioconductor (Dunning et al., 2007). We performed hierarchical clustering of the data using the MATLAB bioinformatics toolbox with Euclidean distance metric and average linkage to generate the hierarchical tree. Data rows (genes) were normalized so that the mean was 0 and the standard deviation was 1. Gene expression data has been deposited into the GEO repository, accession number GSE78864.

Phosphotyrosine proteomic analysis was performed as previously described (Iwai et al., 2013) with the following modifications: SILAC labelled cells (biological triplicates) were lysed in 8M urea and equal amounts of heavy (DasR or PasR cells) and light (parental cells) lysates were mixed prior to reduction, alkylation and trypsin digestion. Peptides were desalted on a C18 cartridge, eluted with 25% acetonitrile and lyophilised to dryness. A two-step enrichment of phosphotyrosine peptides was performed; immunoprecipitation (IP) using a combination of pTyr100, pTyr1000 (Cell Signalling) and 4G10 (Millipore) followed by immobilized metal affinity chromatography (IMAC) on FeCl₃ charged NTA beads as previously described (Iwai et al 2013). Eluted peptides were then subjected to reverse-phase liquid chromatography separation (Iwai et al 2013) followed by electrospray ionization and MS/MS on a Triple-TOF 5600+ mass spectrometer (ABSciex) operated in a data-dependent acquisition mode with top 25 most intense peaks (two to five positive charges) automatically acquired with previously selected peaks excluded for 30 s.

The data were processed with MaxQuant (Cox and Mann, 2008) (version 1.5.2.8) and the peptides were identified (maximal mass error =0.006 Da and 40 ppm for precursor and product ions, respectively) from the MS/MS spectra searched against human referenced proteome (UniProt, June 2015) using

Andromeda (Cox et al., 2011) search engine. The following peptide bond cleavages: arginine or lysine followed by any amino acid (a general setting referred to as Trypsin/P) and up to two missed cleavages were allowed. SILAC based experiments in MaxQuant were performed using the built-in quantification algorithm (Cox and Mann 2008) with minimal ratio count=2 and enabled ‘Re-quantify’ feature. Cysteine carbamidomethylation was selected as a fixed modification whereas methionine oxidation, acetylation of protein N-terminus and phospho (STY) as variable modifications. The false discovery rate was set to 0.01 for peptides, proteins and sites. Other parameters were used as pre-set in the software. “Unique and razor peptides” mode was selected to allow identification and quantification of proteins in groups.

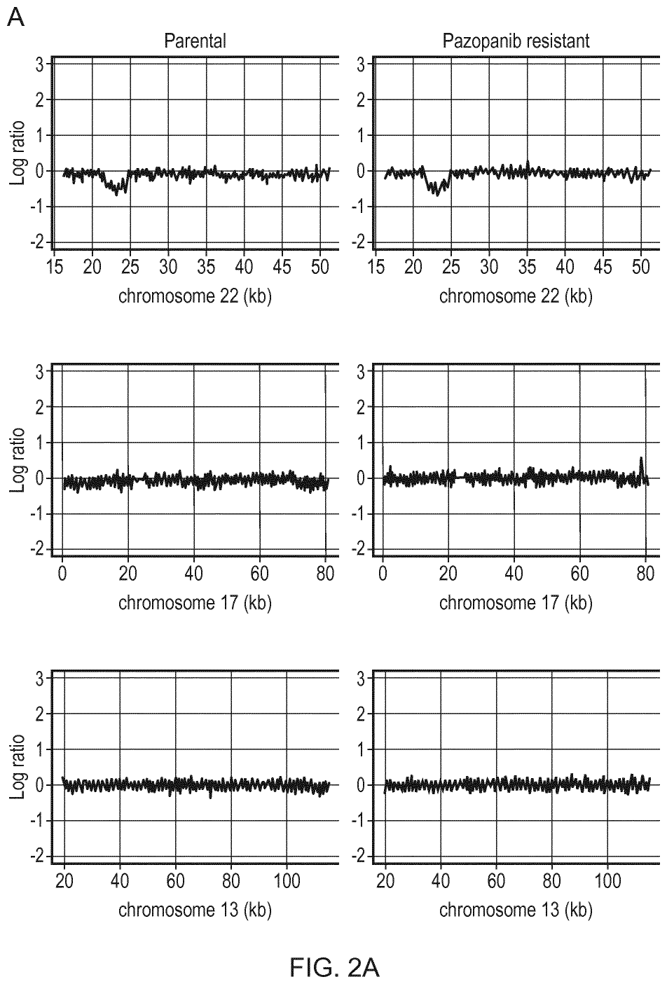

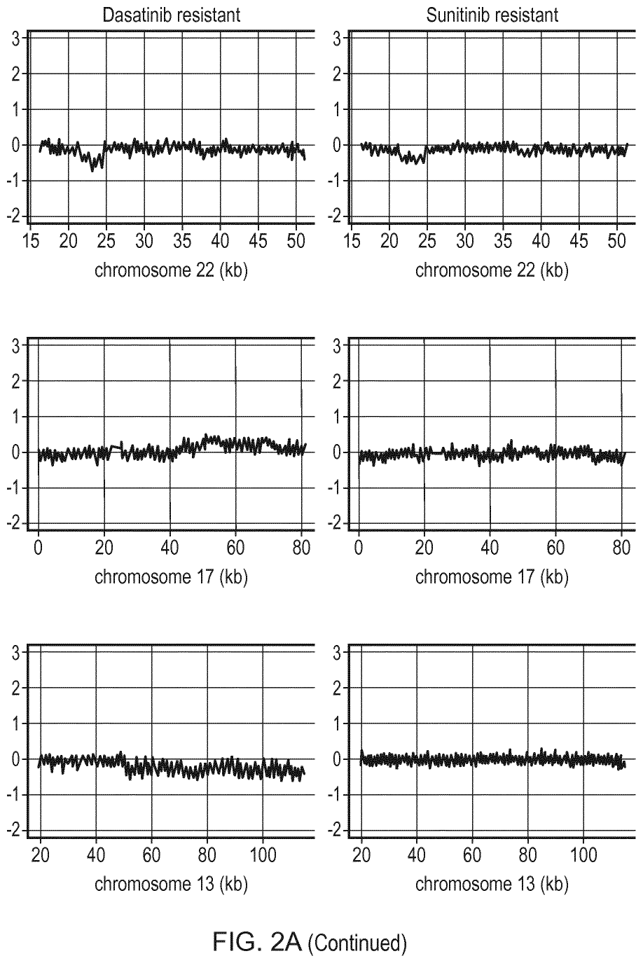

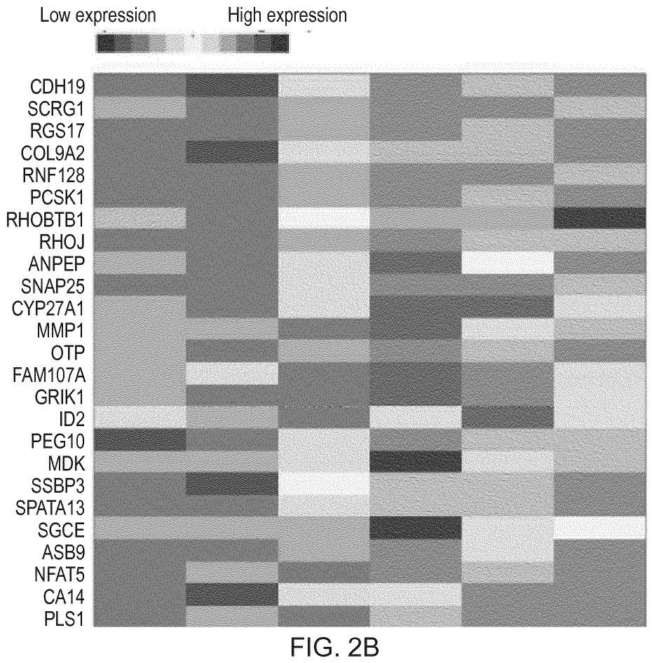

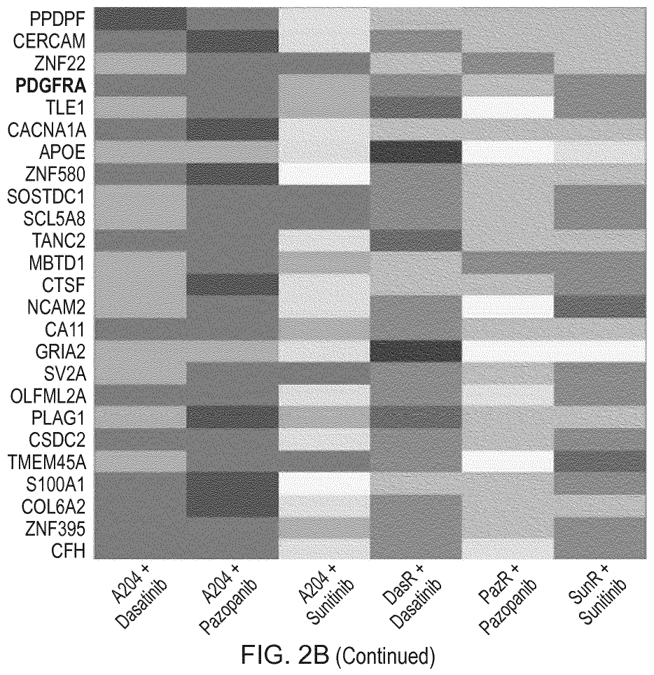

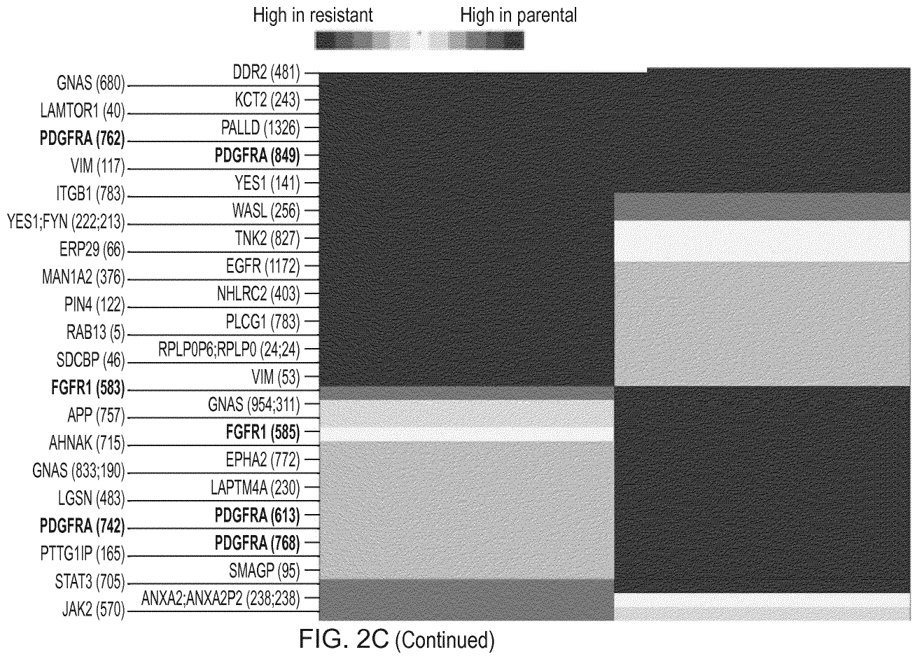

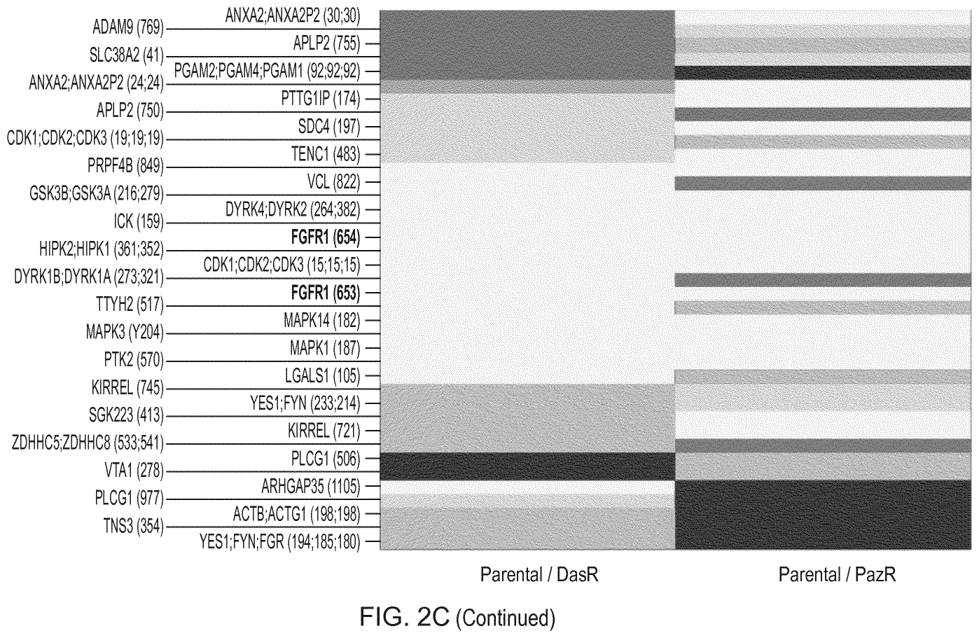

Data were further analysed using Microsoft Office Excel 2007 and Perseus (version 1.5.0.9). The data were filtered to remove potential contaminants and IDs originating from reverse decoy sequences. The log2 values of the heavy/light (H/L) ratios were then determined. An arbitrary value of +10 or −10 was manually imputed when only H or L intensity, respectively, was detected and thus the H/L ratio could not have been automatically assigned by MaxQuant. The data were then normalized to the average H/L ratio of the total proteome (IP supernatant) and filtered to include only high confidence phosphosite IDs (localization probability and score difference ≥90% and 10, respectively). For generation of the heat map (FIG. 2C), normalized H/L ratios of respective triplicates were averaged and reversed (L/H) to visualize the log2 fold changes in phosphorylation between parental (L) and resistant (H) cells.

### Example 1

### MRT Cell Lines are Selectively Responsive to Dasatinib, Pazopanib and Sunitinib

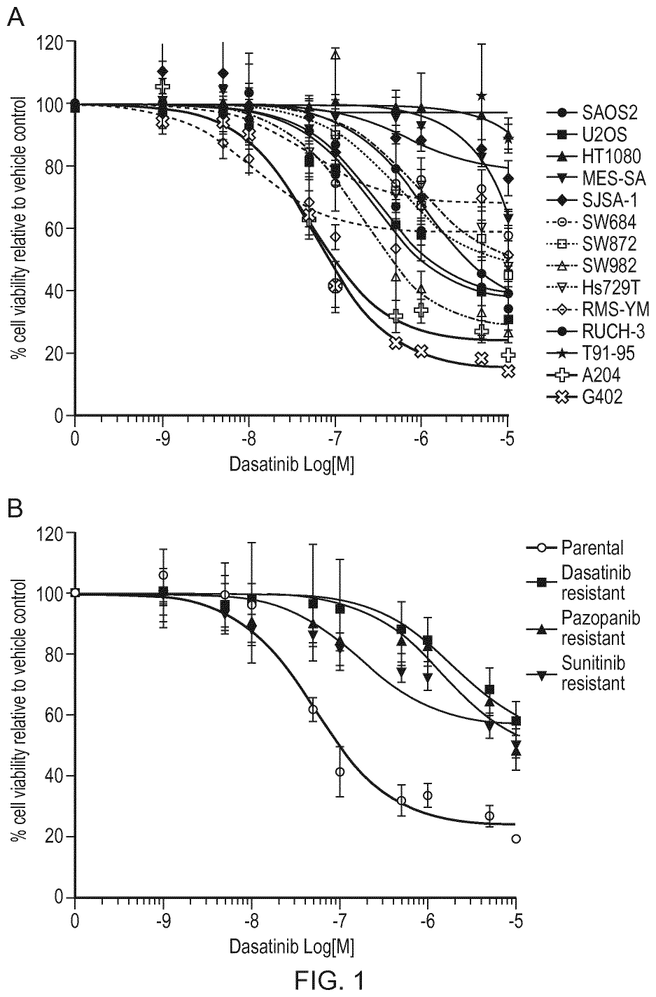

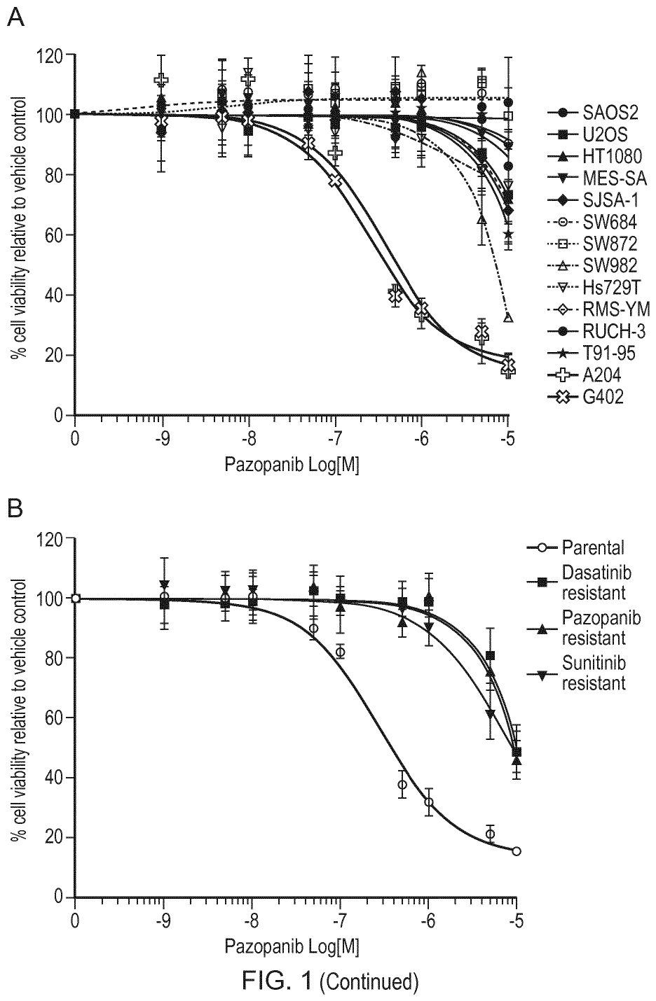

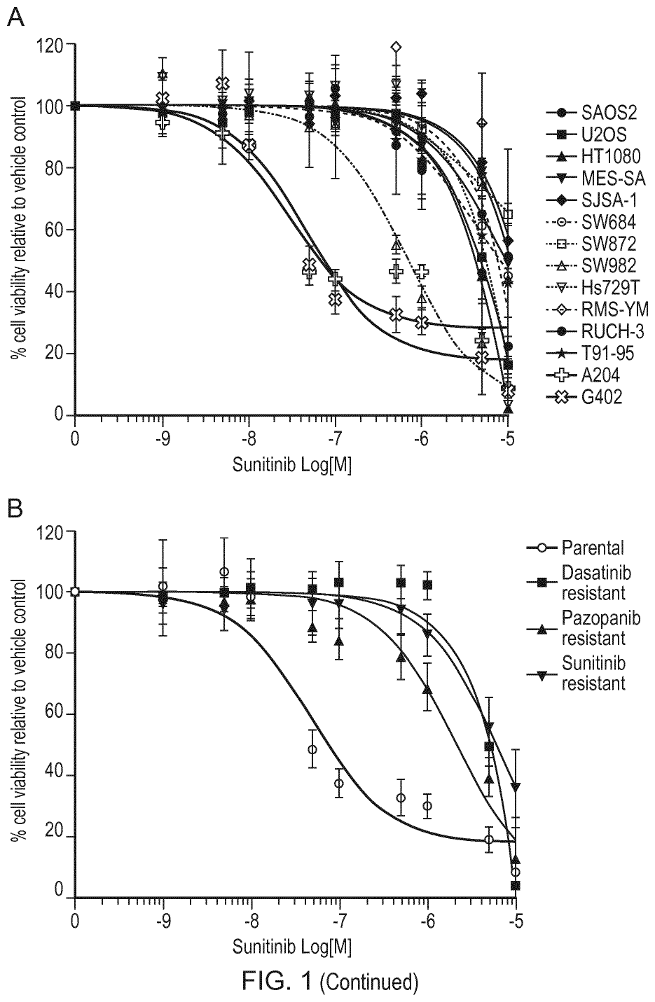

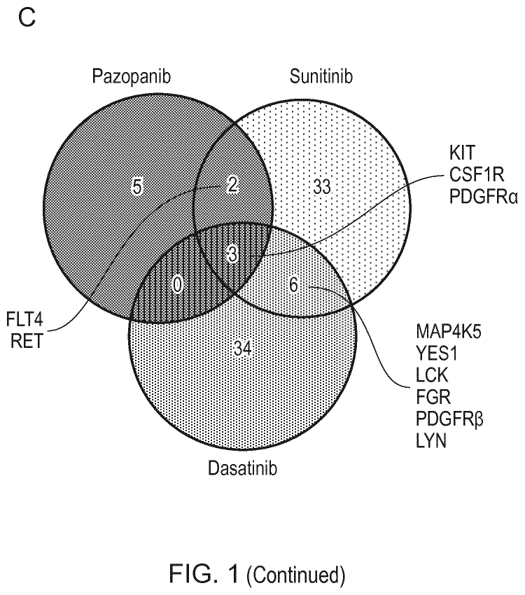

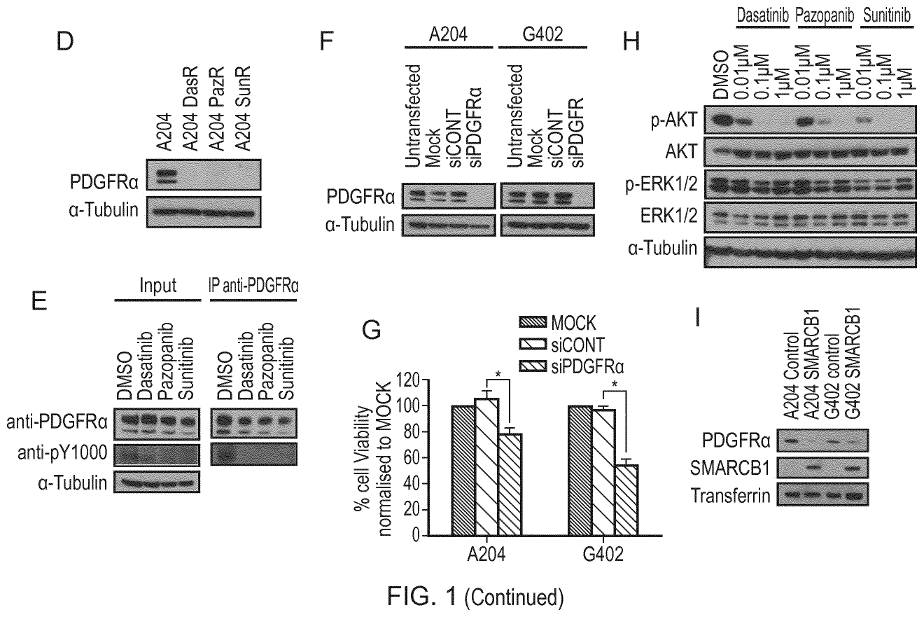

The TKIs dasatinib, pazopanib and sunitinib are either approved or currently being evaluated for soft tissue malignancies such as sarcomas and MRTs. To identify subtypes which may be selectively responsive to these TKIs, a panel of 14 sarcoma and MRT lines were subjected to dose response assessment. Only the MRT cell lines A204 and G402 were found to be sensitive to all three TKIs (FIG. 1A & Table S1).

TABLE S1
Dasatinib, Pazopanib, Sunitinib IC50 concentrations
in a panel of 14 cell lines.

| | Dasatinib IC50 | Pazopanib IC50 | Sunitinib IC50 |
|---|---|---|---|
| Cell Line | (nM) | (nM) | (nM) |
| SAOS2 | 1152.3 +/− 311.0 | >10000 | 4569.7 +/− 516.2 |
| U2OS | >10000 | >10000 | >10000 |
| HT1080 | >10000 | >10000 | >10000 |
| MES-SA | >10000 | >10000 | >10000 |
| SJSA-1 | >10000 | >10000 | >10000 |
| SW684 | 62.4 +/− 25.9 | >10000 | >10000 |
| SW872 | 1038 +/− 490.7 | >10000 | >10000 |
| SW982 | 188.3 +/− 64.7 | >10000 | 581.6 +/− 117.0 |
| Hs729T | >10000 | >10000 | >10000 |
| RMS-YM | >10000 | >10000 | >10000 |
| RUCH-3 | >10000 | >10000 | >10000 |
| T91-95 | >10000 | >10000 | >10000 |
| G402 | 62.3 +/− 21.5 | 237.85 +/− 65.1 | 36.9 +/− 26.5 |
| A204 | 41.8 +/− 5.1 | 218.7 +/− 19.6 | 36.3 +/− 5.5 |

The table gives a list of cell lines used in FIG. 1A and their IC50 values.

### Example 2

### Analysis of Acquired Resistance Identifies PDGFRα as an Oncogenic Driver in MRT Cells

Durable responses to TKIs are rare and most patients develop acquired drug resistance (Kasper et al., 2014). To discover potential resistance mechanisms, we modelled acquired resistance in vitro by subjecting the A204 cells to long-term escalating dose treatment with each of the three TKIs. Cell viability analysis confirmed that these sublines have acquired resistance and were cross-resistant to each other (FIG. 1B & Table S2), suggesting a common mechanism of action.

TABLE S2
Dasatinib, Pazopanib, Sunitinib IC50 concentrations
in A204 resistant cell lines.

| | Dasatinib IC50 | Pazopanib IC50 | Sunitinib IC50 |
|---|---|---|---|
| Cell Line | (nM) | (nM) | (nM) |
| Parental | 41.8 +/− 5.0 | 245 +/− 56.4 | 36.3 +/− 5.5 |
| Dasatinib | >10000 | >10000 | 5010.7 +/− 236.7 |
| resistant | | | |
| Pazopanib | >10000 | >10000 | >10000 |
| resistant | | | |
| Sunitinib | >10000 | >10000 | >10000 |
| resistant | | | |

The table gives a list of cell lines used in FIG. 1B and their IC50 values.

To identify candidate kinases that confer TKI sensitivity, we assessed the target selectivity overlap between the three inhibitors based on published screens of TKI selectivity (Anastassiadis et al., 2011; Davis et al., 2011). Pazopanib, dasatinib and sunitinib share three common RTK targets: c-KIT, CSF1R and PDGFRα (FIG. 1C), of which only PDGFRα is activated in the A204 cells as shown by a previous phosphoproteomic screen (Bai et al., 2012). Immunoblotting revealed a reduction in PDGFRα expression in the acquired resistant sublines (FIG. 1D), indicating that a loss in PDGFRα dependency is a potential mechanism of drug resistance.

Treatment of the parental A204 cells with the three TKIs led to a decrease in PDGFRα phosphorylation (FIG. 1E). Furthermore, siRNA depletion of PDFGRα was able to phenocopy the TKI effects and decrease MRT cell viability (FIGS. 1F & G). Immunoblot analysis of downstream signalling components AKT and ERK1/2, which control cell proliferation and survival, show that the TKIs abolished AKT phosphorylation but had no effect on ERK1/2 phosphorylation in the parental cells (FIG. 1H). Upon ectopic expression of SMARCB1 in the MRT cells, PDGFRα levels are decreased compared to control (FIG. 1I), demonstrating that SMARCB1 regulates PDGFRα expression. Collectively, our findings show that PDFGRα is a driver in MRT cells that is regulated by SMARCB1 and can be effectively inhibited using pazopanib, dasatinib and sunitinib.

### Example 3

### Molecular Profiling of A204 Parental and Resistant Cells

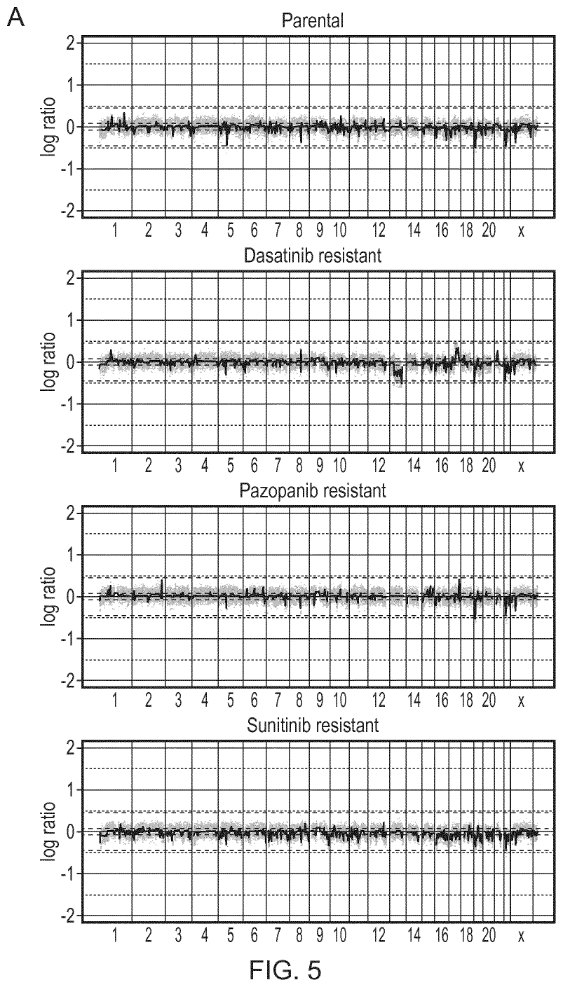

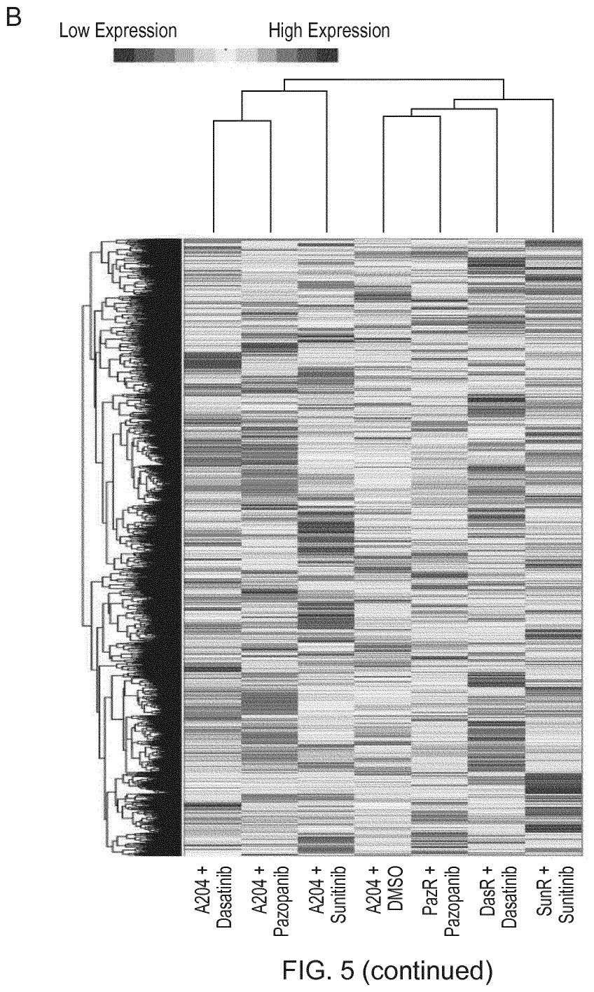

To identify additional candidate drivers in MRTs, we undertook a molecular profiling strategy comprising microarray-based comparative genomic hybridisation (aCGH), gene expression analysis and phosphoproteomics, using the A204 parental and three resistant sublines as a model. aCGH was performed to assess chromosomal gains or losses associated with acquired resistance. The A204 cells have a simple genome with no detectable chromosomal alterations other than a focal deletion of SMARCB1 at 22q11.23 (FIGS. 2A & 5A), which is maintained in the resistant sublines. Of the resistant cells, only the dasatinib resistant (DasR) subline harboured additional gains on chromosome 17q21.32-q25.3 and losses of the whole arm of 13q (FIG. 2A). Since this genomic profile was specific to DasR, it is unlikely that any targets identified in these chromosomal regions will be common to all three TKIs and thus were not pursued further. Gene expression analysis of the four cell lines in the presence of TKI showed that the resistant sublines clustered together with the untreated parental cells (FIG. 5B) and confirmed that PDGFRA was among the most highly downregulated genes in the resistant cells (FIG. 2B).

Phosphoproteomics was used to compare the signalling profiles of DasR and pazopanib resistant (PazR) sublines versus parental cells. Sunitinib resistant (SunR) cells were not analysed because its low proliferation rate prevented sufficient cells from being harvested. We show that parental cells display high levels of phosphorylated PDGFRα at multiple sites (Y613, Y742, Y762, Y768 and Y849) (FIG. 2C). Interestingly, FGFR1 phosphorylation in the kinase insert domain (Y583 and Y585) was also found to be elevated in the parental cells. Additionally, FGFR1 was phosphorylated in its activation loop (Y653 and Y654) at similar levels in both parental and resistant cells. This data confirms that PDGFRα is the only common kinase target of pazopanib, dasatinib and sunitinib that is activated in these cells (FIG. 1C) and demonstrates that both PDGFRα and FGFR1 are coactivated with multiple phosphosites observed in each receptor.

### Example 4

### Dual Targeting of PDGFRα and FGFR1 Enhances Apoptosis

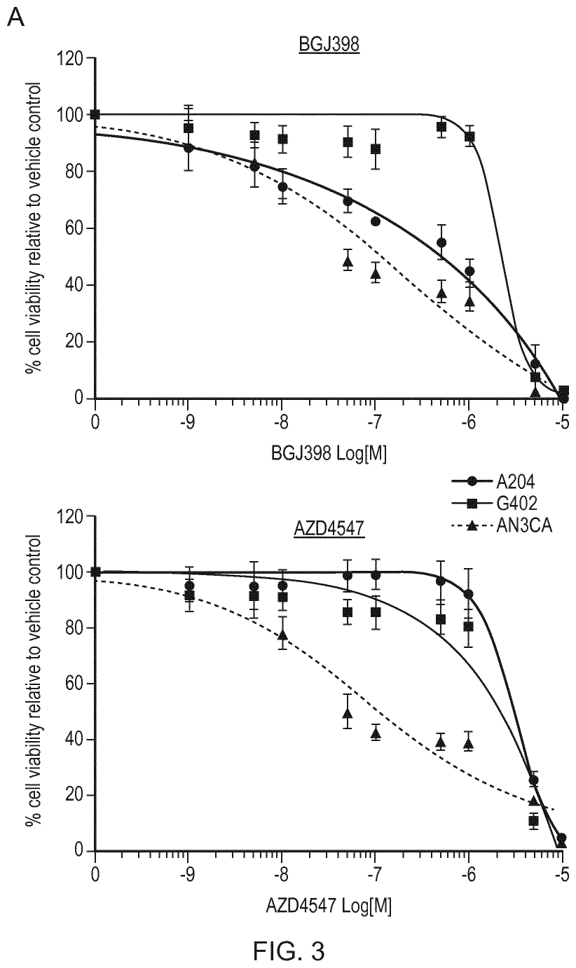

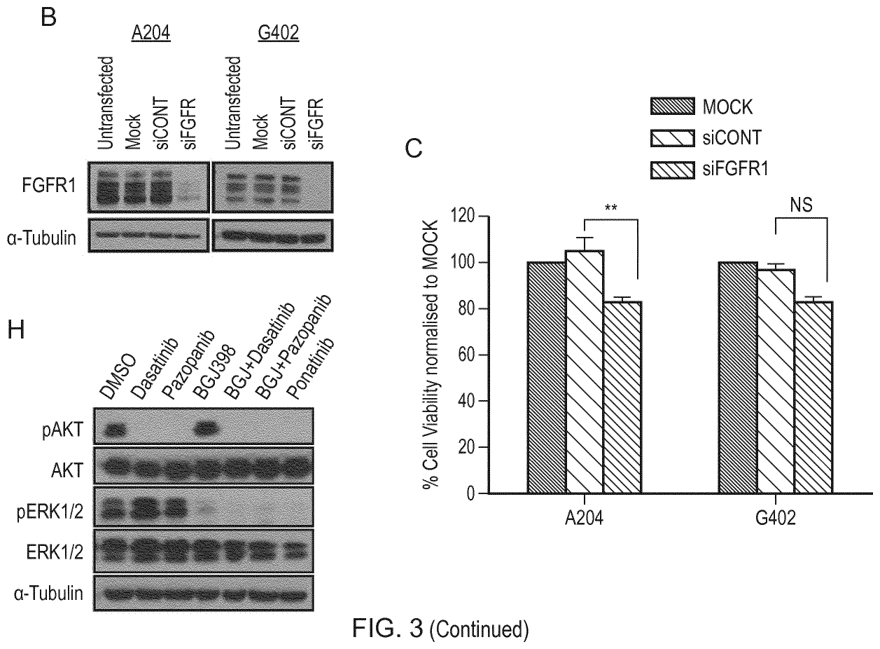

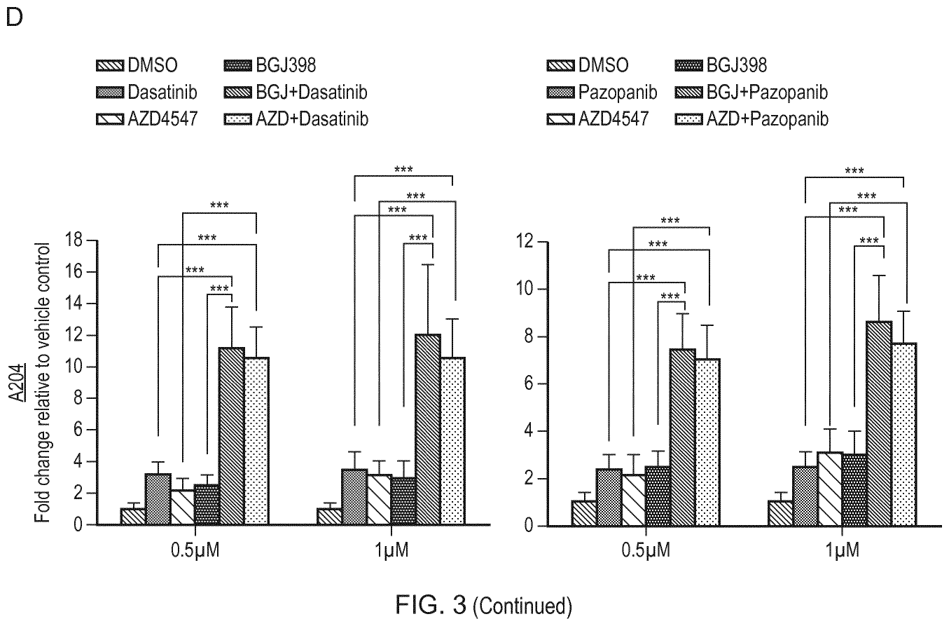

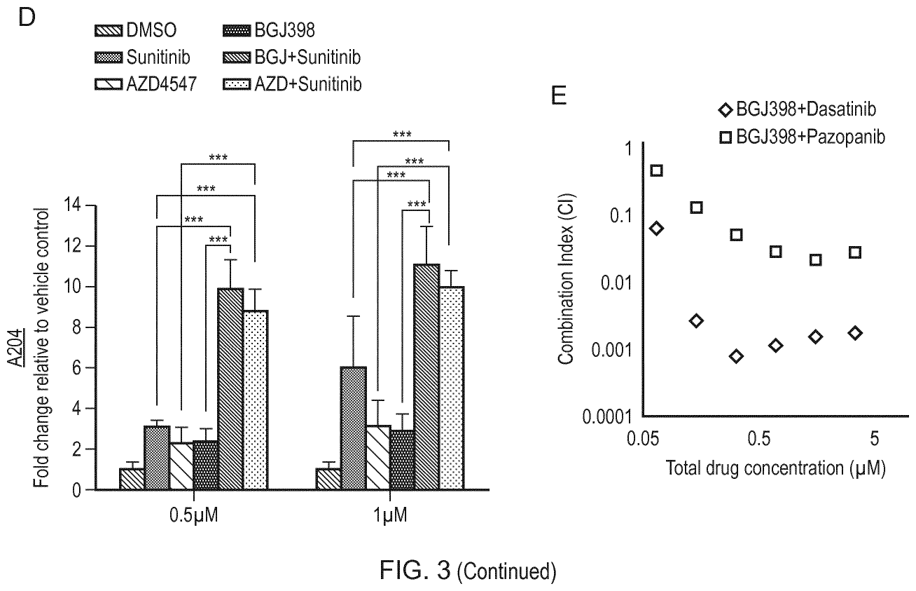

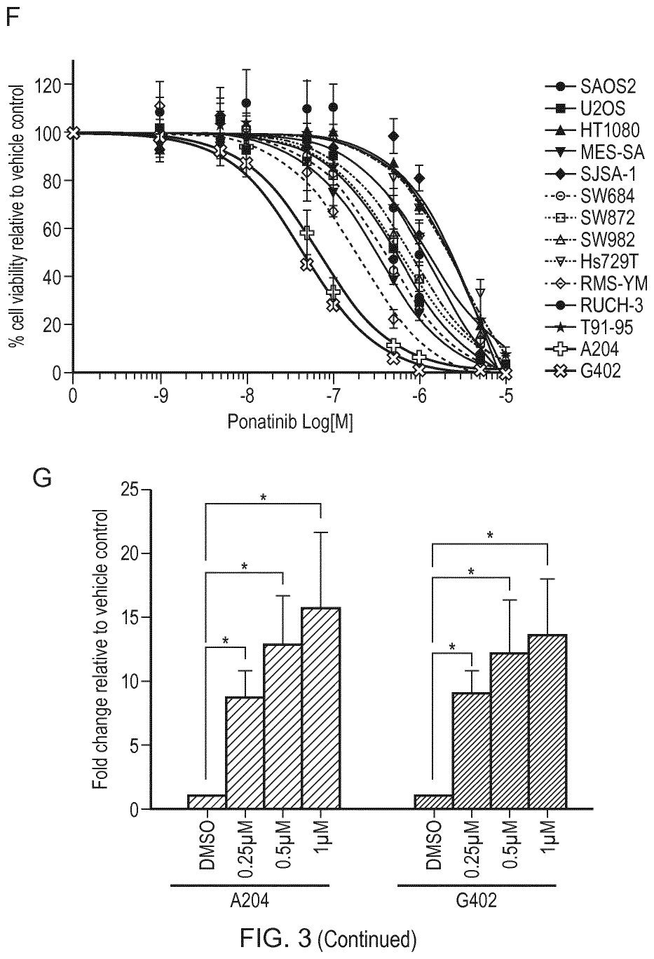

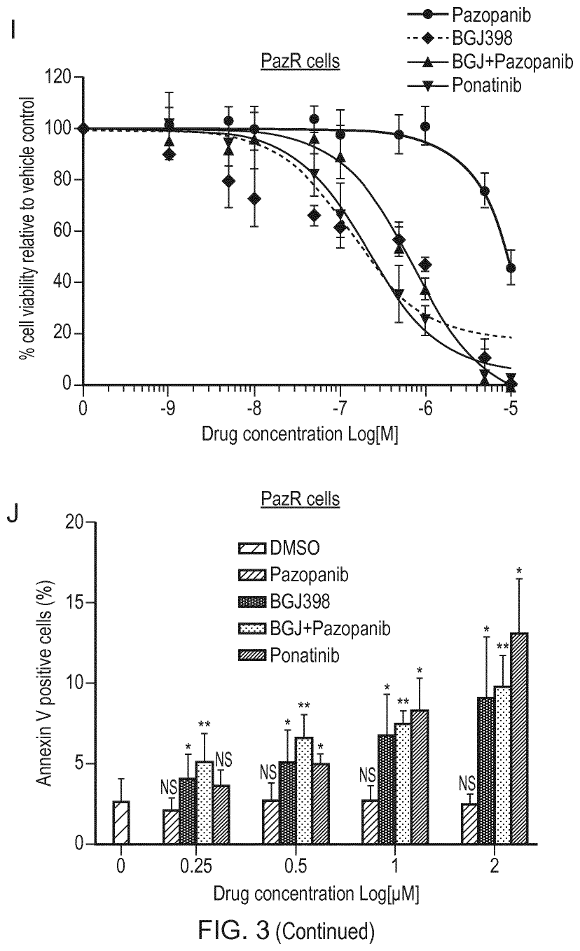

FGFR RTKs are therapeutic targets in MRTs (Wohrle et al., 2013), so following the uncovering of FGFR1 phosphorylation in our phosphoproteomic analysis, we assessed the effects of two selective FGFR TKIs NVP-BGJ398 and AZD4547 on the viability of A204 and G402 cells (Tan et al., 2014). AZD4547 was ineffective in both cell lines while BGJ398 only reduced viability in the A204 cells (FIG. 3A). As a positive control, AN3CA cells which harbour an FGFR2 mutation and are sensitive to FGFR TKIs was used (Tan et al., 2014). Depletion of FGFR1 using siRNA also showed a minor decrease in the viability of the MRT cells (FIGS. 3B & C).

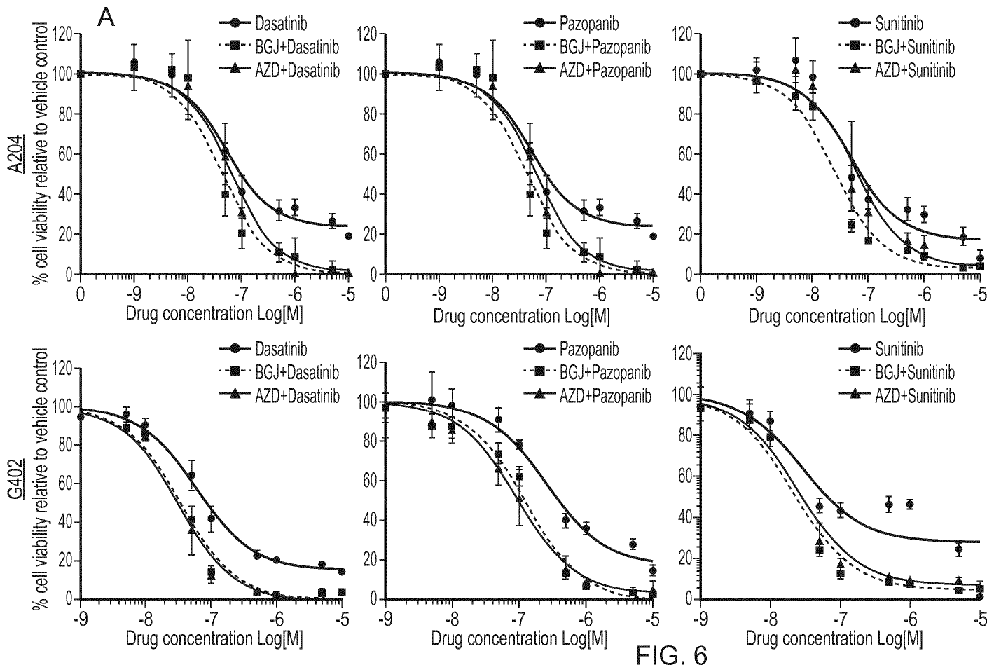

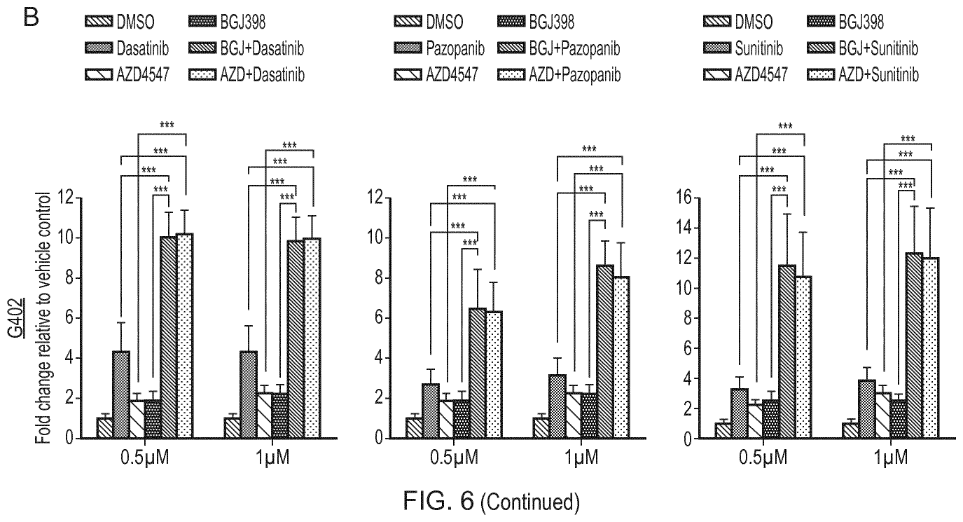

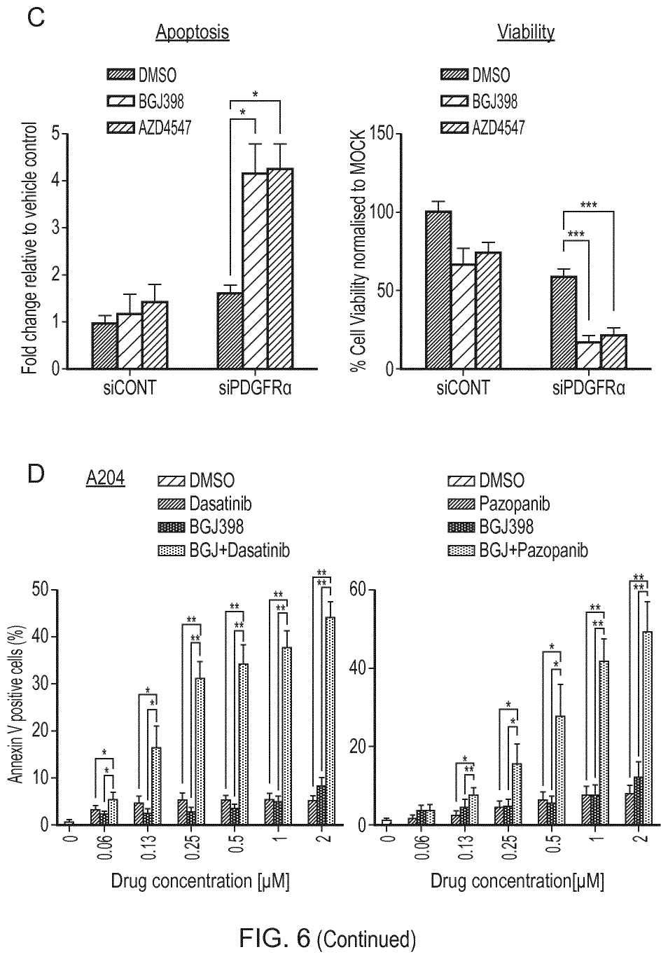

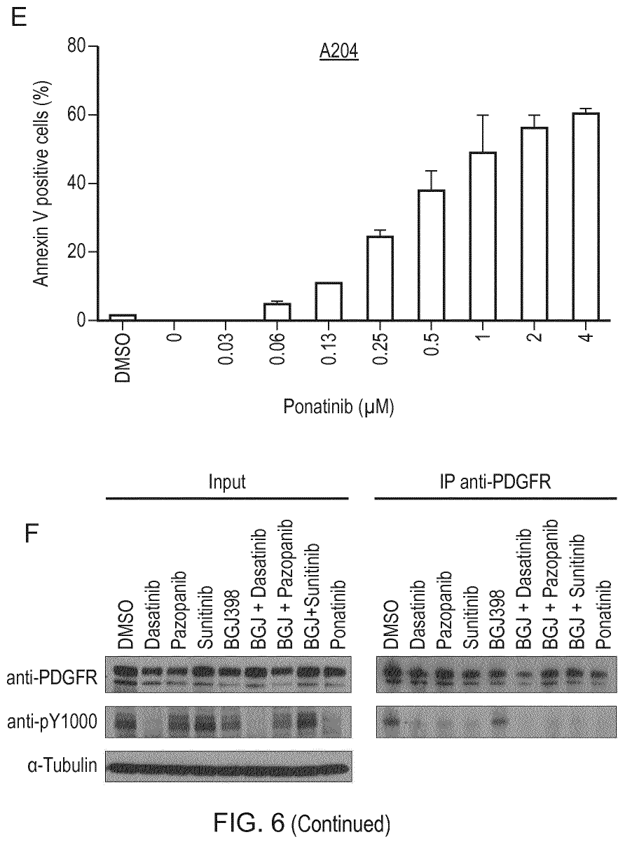

We evaluated the effects of BGJ398 and AZD4547 in combination with PDGFRα TKIs on cell viability and apoptosis. This combination showed a small decrease in A204 and G402 viability compared to single inhibitor treatment (FIG. 6A) reflecting the strong cytostatic consequence of PDGFRα TKI monotherapy (FIG. 1A). Assessment of caspase 3/7 activity finds that PDGFRα or FGFR TKI treatment alone led to low levels of apoptosis despite high drug concentrations of up to 1 μM (FIGS. 3D & 6B). Dual PDGFRα and FGFR inhibition showed significantly increased apoptosis (>6-fold relative to vehicle control). This enhanced apoptosis was recapitulated with a combination of siRNA depletion of PDGFRα and BGJ398 or AZD4547 treatment (FIG. 6C). To assess if the combination confers synergistic cytotoxicity in the A204 cells, we employed an automated imaging assay to visualise annexin V positive cells. While the individual TKIs only resulted in <5% apoptotic cells (FIG. 6D), the combination of BGJ398 with either pazopanib or dasatinib led to a synergistic increase (combination index <1) in the proportion of apoptotic cells to ˜30-50% across all drug doses tested (FIGS. 3E & 6D).

To establish if a dual inhibitor of both receptors is capable of inducing apoptosis as a single agent, the effects of ponatinib, a potent inhibitor of FGFR1 and PDGFRα (Gozgit et al., 2011), was investigated. While previous reports claim that pazopanib and sunitinib are FGFR1 inhibitors, the K_{D} of these compounds for FGFR1 are 128-fold and 67-fold higher respectively compared to ponatinib (Tucker et al., 2014). Assessing the dose response effects of ponatinib in the panel of 14 cell lines confirms that the MRT cell lines are sensitive to this TKI (FIG. 3F). Treatment with ponatinib resulted in enhanced apoptosis in the MRT cells, at levels similar to combined PDGFRα and FGFR TKI treatment (FIGS. 3G & 6E).

In contrast to the PDGFRα TKIs, FGFR inhibitor (BGJ398) treatment had no effect on AKT phosphorylation but instead decreased ERK1/2 phosphorylation (FIG. 3H). As expected, BGJ398 had no effects on PDGFRα phosphorylation (FIG. 6F). Correspondingly, combined treatment with PDGFRα and FGFR TKIs or ponatinib resulted in the suppression of both ERK1/2 and AKT phosphorylation (FIG. 3H), consistent with a model where inhibition of both pathways is required for inducing apoptosis in MRT cells.

### Example 5

### FGFR Inhibitors Sensitize MRT Cells that have Acquired Resistance to Pazopanib

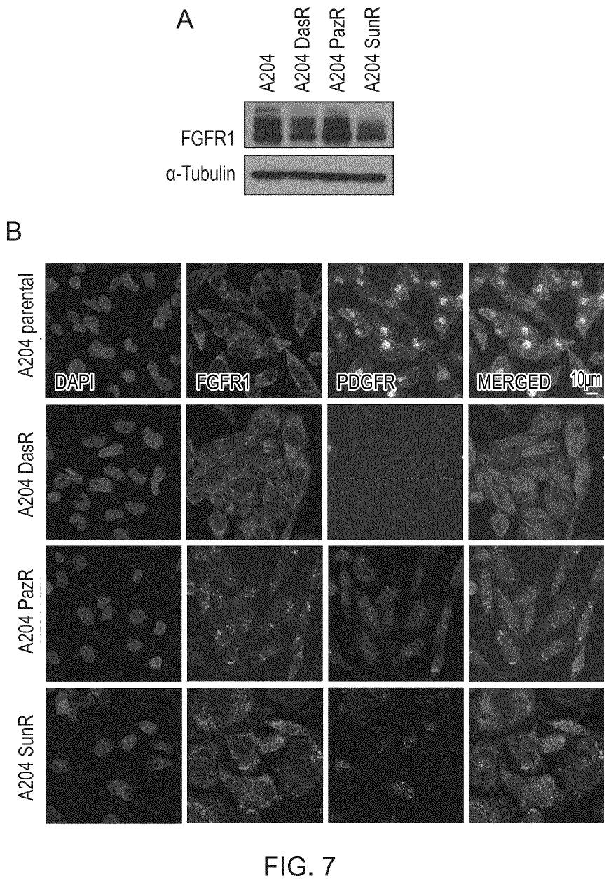

Given that pazopanib is approved for soft tissue malignancies and there is currently no effective means to treat patients whose tumours have progressed on this TKI, we investigated if targeting FGFR1 is capable of sensitizing cells that have acquired pazopanib resistance. The resistant sublines maintain FGFR1 expression (FIG. 7A) and activation loop phosphorylation (FIG. 2C) at similar levels as the parental cells. Treating PazR cells with BGJ398 led to a reduction in cell viability which was not enhanced by the addition of pazopanib, demonstrating that these cells are no longer addicted to PDGFRα (FIG. 3I and Table S3). The degree of sensitization of the pazopanib resistant cells in response to BGJ398 was similar to the IC₅₀ of pazopanib treatment in the parental A204 cells (Table S2). Pazopanib alone had no effect on apoptosis compared to vehicle control while BGJ398, ponatinib or the combination of BGJ398 and pazopanib led to a significant increase in proportion of apoptotic cells (FIG. 3J). This data demonstrates that FGFR1 blockade is an effective means of overcoming resistance to pazopanib.

TABLE S3
Single and combination drug treatment IC50 concentrations
in Pazopanib resistant A204 cell line.

| | IC50 |
|---|---|
| Drug treatment | (nM) |
| Pazopanib | >10000 |
| BGJ398 | 247.4 +/− 29.2 |
| BGJ398 + Pazopanib | 690.1 +/− 133.1 |
| Ponatinib | 271.5 +/− 167.8 |

The table gives drug treatments used in FIG. 3I and their IC50 values.

In some cancers subpopulations of cancer cells display mutually exclusive RTK amplification events reflecting intratumoural heterogeneity, and clonal selection during therapy leads to acquired resistance (Szerlip et al., 2012). Previous FISH analysis of A204 cells finds that PDGFRα is not amplified at the genomic level (McDermott et al., 2009). To establish if heterogeneity in RTK expression could be a potential mechanism for drug resistance, immunofluorescence was performed to determine the distribution of PDGFRα and FGFR1. We find that both RTKs are expressed in all cells within the parental A204 population (FIG. 7B) and consistent with the immunoblot data, the three resistant sublines display reduced PDGFRα levels and maintain FGFR1 expression. This data confirms that RTK expression is not mutually exclusive in distinct subpopulations and suggests that acquired resistance is unlikely the result of clonal selection of a pre-existing PDGFRα-deficient subpopulation but rather the consequence of genetic evolution by PDGFRα loss in drug tolerant cells during TKI selection (Hata et al., 2016).

### Discussion

We show that PDGFRα levels are regulated by SMARCB1. MRT cells that have acquired resistance to the PDGFRα inhibitor pazopanib are susceptible to FGFR inhibitors.

Dual blockade of both RTKs promotes cytotoxicity across all drug doses tested in A204 and G402 cells. Inhibitor combinations targeting both receptors and the dual inhibitor ponatinib suppresses the AKT and ERK1/2 pathways leading to apoptosis. We show that ponatinib, a dual PDGFRα and FGFR1 inhibitor, induces apoptosis in MRT cells as a single agent.

Wohrle et al. showed that FGFR1 is upregulated when SMARCB1 is deleted in MRT cells (Wohrle et al., 2013). By showing that SMARCB1 loss also regulates PDGFRα expression levels, our study provides further evidence that exploiting RTK dependencies in cancers driven by SWI/SNF deficiencies is an effective therapeutic strategy. Since it is currently not possible to directly target the SWI/SNF complex, TKI combinations may have broader clinical utility in the treatment of this class of cancers.

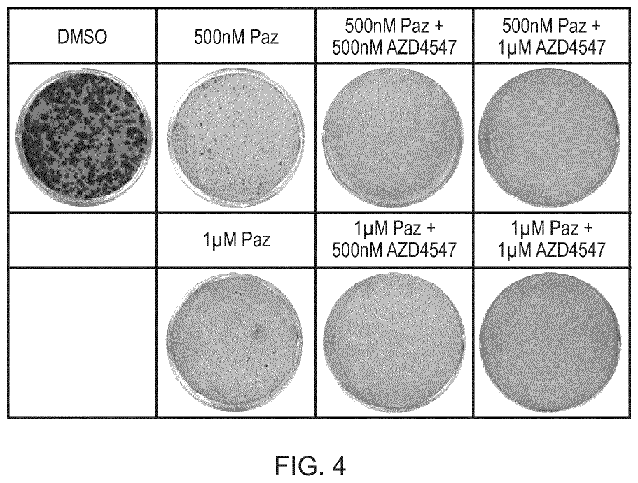

Since it is less likely for cancer cells to develop acquired resistance when multiple RTKs are simultaneously inhibited upfront, there is a rationale for using the PDGFRα and FGFR1 inhibitor combination as first line therapy. Indeed, attempts to generate acquired resistant lines to the PDGFRα and FGFR inhibitor combination have been unsuccessful (FIG. 4).

In summary, we show that MRTs are exquisitely sensitive to the combined inhibition of PDGFRα and FGFR1 and that ponatinib is effective as a single agent in this disease. Treatment with FGFR inhibitors sensitizes MRT cells that have acquired resistance to pazopanib.

### REFERENCES

Anastassiadis, T., Deacon, S. W., Devarajan, K., Ma, H., and Peterson, J. R. (2011). Comprehensive assay of kinase catalytic activity reveals features of kinase inhibitor selectivity. Nat Biotechnol 29, 1039-1045.
Bai, Y., Li, J., Fang, B., Edwards, A., Zhang, G., Bui, M., Eschrich, S., Altiok, S., Koomen, J., and Haura, E. B. (2012). Phosphoproteomics identifies driver tyrosine kinases in sarcoma cell lines and tumors. Cancer Res 72, 2501-2511.
Davis, M. I., Hunt, J. P., Herrgard, S., Ciceri, P., Wodicka, L. M., Pallares, G., Hocker, M., Treiber, D. K., and Zarrinkar, P. P. (2011). Comprehensive analysis of kinase inhibitor selectivity. Nat Biotechnol 29, 1046-1051.
Gozgit, J. M., Wong, M. J., Wardwell, S., Tyner, J. W., Loriaux, M. M., Mohemmad, Q. K., Narasimhan, N. I., Shakespeare, W. C., Wang, F., Druker, B. J., et al. (2011). Potent activity of ponatinib (AP24534) in models of FLT3-driven acute myeloid leukemia and other hematologic malignancies. Mol Cancer Ther 10, 1028-1035.
Hata, A. N., Niederst, M. J., Archibald, H. L., Gomez-Caraballo, M., Siddiqui, F. M., Mulvey, H. E., Maruvka, Y. E., Ji, F., Bhang, H. C., Krishnamurthy Radhakrishna, V., et al. (2016). Tumor cells can follow distinct evolutionary paths to become resistant to epidermal growth factor receptor inhibition. Nat Med.
Huang, P. H., Mukasa, A., Bonavia, R., Flynn, R. A., Brewer, Z. E., Cavenee, W. K., Furnari, F. B., and White, F. M. (2007). Quantitative analysis of EGFRvIII cellular signaling networks reveals a combinatorial therapeutic strategy for glioblastoma. Proc Natl Acad Sci USA 104, 12867-12872.
Iwai, L. K., Payne, L. S., Luczynski, M. T., Chang, F., Xu, H., Clinton, R. W., Paul, A., Esposito, E. A., Gridley, S., Leitinger, B., et al. (2013). Phosphoproteomics of collagen receptor networks reveals SHP-2 phosphorylation downstream of wild-type DDR2 and its lung cancer mutants. Biochem J 454, 501-513.
Kadoch, C., and Crabtree, G. R. (2013). Reversible disruption of mSWI/SNF (BAF) complexes by the SS18-SSX oncogenic fusion in synovial sarcoma. Cell 153, 71-85.
Kadoch, C., Hargreaves, D. C., Hodges, C., Elias, L., Ho, L., Ranish, J., and Crabtree, G. R. (2013). Proteomic and bioinformatic analysis of mammalian SWI/SNF complexes identifies extensive roles in human malignancy. Nat Genet 45, 592-601.
Kasper, B., Sleijfer, S., Litiere, S., Marreaud, S., Verweij, J., Hodge, R. A., Bauer, S., Kerst, J. M., and van der Graaf, W. T. (2014). Long-term responders and survivors on pazopanib for advanced soft tissue sarcomas: subanalysis of two European Organisation for Research and Treatment of Cancer (EORTC) clinical trials 62043 and 62072. Ann Oncol 25, 719-724.
Kim, K. H., and Roberts, C. W. (2014). Mechanisms by which SMARCB1 loss drives rhabdoid tumor growth. Cancer Genet 207, 365-372.
Lee, R. S., Stewart, C., Carter, S. L., Ambrogio, L., Cibulskis, K., Sougnez, C., Lawrence, M. S., Auclair, D., Mora, J., Golub, T. R., et al. (2012). A remarkably simple genome underlies highly malignant pediatric rhabdoid cancers. J Clin Invest 122, 2983-2988.
Lemmon, M. A., and Schlessinger, J. (2010). Cell signaling by receptor tyrosine kinases. Cell 141, 1117-1134.
Madigan, C. E., Armenian, S. H., Malogolowkin, M. H., and Mascarenhas, L. (2007). Extracranial malignant rhabdoid tumors in childhood: the Childrens Hospital Los Angeles experience. Cancer 110, 2061-2066.
McDermott, U., Ames, R. Y., Iafrate, A. J., Maheswaran, S., Stubbs, H., Greninger, P., McCutcheon, K., Milano, R., Tam, A., Lee, D. Y., et al. (2009). Ligand-dependent platelet-derived growth factor receptor (PDGFR)-alpha activation sensitizes rare lung cancer and sarcoma cells to PDGFR kinase inhibitors. Cancer Res 69, 3937-3946.
Papadakis, A. I., Sun, C., Knijnenburg, T. A., Xue, Y., Grernrum, W., Holzel, M., Nijkamp, W., Wessels, L. F., Beijersbergen, R. L., Bernards, R., et al. (2015). SMARCE1 suppresses EGFR expression and controls responses to MET and ALK inhibitors in lung cancer. Cell Res 25, 445-458.
Szerlip, N. J., Pedraza, A., Chakravarty, D., Azim, M., McGuire, J., Fang, Y., Ozawa, T., Holland, E. C., Huse, J. T., Jhanwar, S., et al. (2012). Intratumoral heterogeneity of receptor tyrosine kinases EGFR and PDGFRA amplification in glioblastoma defines subpopulations with distinct growth factor response. Proc Natl Acad Sci USA 109, 3041-3046.
Tan, L., Wang, J., Tanizaki, J., Huang, Z., Aref, A. R., Rusan, M., Zhu, S. J., Zhang, Y., Ercan, D., Liao, R. G., et al. (2014). Development of covalent inhibitors that can overcome resistance to first-generation FGFR kinase inhibitors. Proc Natl Acad Sci USA 111, E4869-4877.
Tucker, J. A., Klein, T., Breed, J., Breeze, A. L., Overman, R., Phillips, C., and Norman, R. A. (2014). Structural insights into FGFR kinase isoform selectivity: diverse binding modes of AZD4547 and ponatinib in complex with FGFR1 and FGFR4. Structure 22, 1764-1774.
Wohrle, S., Weiss, A., Ito, M., Kauffmann, A., Murakami, M., Jagani, Z., Thuery, A., Bauer-Probst, B., Reimann, F., Stamm, C., et al. (2013). Fibroblast growth factor receptors as novel therapeutic targets in SNF5-deleted malignant rhabdoid tumors. PLoS One 8, e77652.
Xu, A. M., and Huang, P. H. (2010). Receptor tyrosine kinase coactivation networks in cancer. Cancer Res 70, 3857-3860.
Anders, S., Pyl, P. T., and Huber, W. (2015). HTSeq—a Python framework to work with high-throughput sequencing data. Bioinformatics 31, 166-169.
Coe, B. P., Ylstra, B., Carvalho, B., Meijer, G. A., Macaulay, C., and Lam, W. L. (2007). Resolving the resolution of array CGH. Genomics 89, 647-653.
Cox, J., and Mann, M. (2008). MaxQuant enables high peptide identification rates, individualized p.p.b.-range mass accuracies and proteome-wide protein quantification. Nat Biotechnol 26, 1367-1372.
Cox, J., Neuhauser, N., Michalski, A., Scheltema, R. A., Olsen, J. V., and Mann, M. (2011). Andromeda: a peptide search engine integrated into the MaxQuant environment. J Proteome Res 10, 1794-1805.
Dobin, A., Davis, C. A., Schlesinger, F., Drenkow, J., Zaleski, C., Jha, S., Batut, P., Chaisson, M., and Gingeras, T. R. (2013). STAR: ultrafast universal RNA-seq aligner. Bioinformatics 29, 15-21.
Dunning, M. J., Smith, M. L., Ritchie, M. E., and Tavare, S. (2007). beadarray: R classes and methods for Illumina bead-based data. Bioinformatics 23, 2183-2184.
Gunnarsson, R., Staaf, J., Jansson, M., Ottesen, A. M., Goransson, H., Liljedahl, U., Ralfkiaer, U., Mansouri, M., Buhl, A. M., Smedby, K. E., et al. (2008). Screening for copy-number alterations and loss of heterozygosity in chronic lymphocytic leukemia—a comparative study of four differently designed, high resolution microarray platforms. Genes Chromosomes Cancer 47, 697-711.
Iwai, L. K., Payne, L. S., Luczynski, M. T., Chang, F., Xu, H., Clinton, R. W., Paul, A., Esposito, E. A., Gridley, S., Leitinger, B., et al. (2013). Phosphoproteomics of collagen receptor networks reveals SHP-2 phosphorylation downstream of wild-type DDR2 and its lung cancer mutants. Biochem J 454, 501-513.
Love, M. I., Huber, W., and Anders, S. (2014). Moderated estimation of fold change and dispersion for RNA-seq data with DESeq2. Genome Biol 15, 550.
Marchio, C., Iravani, M., Natrajan, R., Lambros, M. B., Savage, K., Tamber, N., Fenwick, K., Mackay, A., Senetta, R., Di Palma, S., et al. (2008). Genomic and immunophenotypical characterization of pure micropapillary carcinomas of the breast. J Pathol 215, 398-410.
Natrajan, R., Lambros, M. B., Rodriguez-Pinilla, S. M., Moreno-Bueno, G., Tan, D. S., Marchio, C., Vatcheva, R., Rayter, S., Mahler-Araujo, B., Fulford, L. G., et al. (2009). Tiling path genomic profiling of grade 3 invasive ductal breast cancers. Clin Cancer Res 15, 2711-2722.
Natrajan, R., Wilkerson, P. M., Marchio, C., Piscuoglio, S., Ng, C. K., Wai, P., Lambros, M. B., Samartzis, E. P., Dedes, K. J., Frankum, J., et al. (2014). Characterization of the genomic features and expressed fusion genes in micropapillary carcinomas of the breast. J Pathol 232, 553-565.
Todd, J. R., Scurr, L. L., Becker, T. M., Kefford, R. F., and Rizos, H. (2014). The MAPK pathway functions as a redundant survival signal that reinforces the PI3K cascade in c-Kit mutant melanoma. Oncogene 33, 236-245.
Modena, P., Lualdi, E., Facchinetti, F., Galli, L., Teixeira, M. R., Pilotti, S., and Sozzi, G. (2005). SMARCB1/INI1 Tumor Suppressor Gene Is Frequently Inactivated in Epithelioid Sarcomas. Cancer Res 65, 4012-4019. May 15, 2005.
Patani, H., Bunney, T. D., Thiyagarajan, N., Norman, R. A., Ogg, D., Breed, J., Ashford, P., Potterton, A., Edwards, M., Williams, S. V., Thomson, G. S., Pang, C. S. M., Knowles, M. A., Breeze, A. L., Orengo, C., Phillips, C., and Katan, M. (2016) Landscape of activating cancer mutations in FGFR kinases and their differential responses to inhibitors in clinical use. Oncotarget 7, 17, March 2016 24252-24268
K. Kohashi, Y. Oda, H. Yamamoto, S. Tamiya, H. Matono, Y. Iwamoto, T. Taguchi, M. Tsuneyoshi, Reduced expression of SMARCB1/INI1 protein in synovial sarcoma. Mod Pathol 23, 981 (2010).
B. Rekhi, U. Vogel, Utility of characteristic ‘Weak to Absent’ INI1/SMARCB1/BAF47 expression in diagnosis of synovial sarcomas. APMIS 123, 618 (2015).
Tolcher, A. W., Papadopoulos, K. P., Patnaik, A., Wilson, K., Thayer, S., Zanghi, J., Gemo, A. T., Kavanaugh, W. M., Keer, H. N., LoRusso, P. M., A phase I, first in human study of FP-1039 (GSK3052230), a novel FGF ligand trap, in patients with advanced solid tumors; Ann Oncol. 2016 March; 27(3):526-32. Epub 2015 Dec. 8.

1. A method of treating a SMARCB1 deficient cancer in an individual, comprising administration of an effective amount of an inhibitor of PDGFRα and an inhibitor of FGFR.

2. A method as claimed in claim 1, wherein the cancer is selected from rhabdoid tumour, epithelioid sarcoma, renal medullary carcinoma, epithelioid malignant peripheral nerve sheath tumour, extraskeletal myxoid chondrosarcoma, cribiform neuroepithelial tumour of the ventricle, collecting duct carcinoma and synovial sarcoma.

3. The method of claim 2, wherein the cancer is a rhabdoid tumour such as a malignant rhabdoid tumour (MRT), or an atypical teratoid rhabdoid tumour (AT/RT).

4. (canceled)

5. The method of claim 1, wherein at least one inhibitor is a small molecule inhibitor, an antibody, a ligand trap, a peptide fragment or a nucleic acid inhibitor.

6. The method of claim 1, wherein the PDGFRα inhibitor is selected from pazopanib, olaratumab, lucitanib, ponatinib, dasatinib and sunitinib.

7.-8. (canceled)

9. The method of claim 1, wherein the FGFR inhibitor is an inhibitor of FGFR1, FGFR2, FGFR3 and/or FGFR4 and said inhibitor is selected from NVP-BGJ398, AZD4547, TKI258, JNJ42756493, lucitanib and ponatinib.

10. The method of claim 1, wherein the inhibitor of PDGFRα and inhibitor of FGFR are the same molecule.

11. The method of claim 10, wherein the inhibitor is lucitanib or ponatinib.

12. The method of claim 1, wherein the combined use of the inhibitor of PDGFRα and inhibitor of FGFR produces at least one of
i) a synergistic effect
ii) induction of apoptosis of cancer cells and, or
iii) sensitization of cancer cells to said PDGFRα inhibitor and wherein the inhibitors of PDGFRα and FGFR are administered simultaneously or sequentially.

13.-14. (canceled)

15. The method of claim 1, wherein the cancer is resistant to a PDGFRα inhibitor alone.

16. The method of inhibitor of claim 15, wherein resistance to a PDGFRα inhibitor is determined by tumour growth and/or metastasis after treatment with a PDGFRα inhibitor.

17.-18. (canceled)

19. The method of claim 1, wherein the individual has been determined to have SMARCB1 deficient cancer prior to treatment, as determined by measuring SMARCB1 protein expression in said sample.

20.-23. (canceled)

24. A pharmaceutical composition comprising an inhibitor of PDGFRα and an inhibitor of FGFR in a suitable carrier, wherein the inhibitor of PDGFRα and the inhibitor of FGFR are different.

25.-26. (canceled)

27. A method of treating a pazopanib resistant cancer in an individual comprising administration of a therapeutically effective amount of an FGFR inhibitor to said individual and wherein resistance to pazopanib is determined by tumour growth and/or metastasis after treatment with pazopanib.

28. The method according to claim 27, wherein the cancer is selected from a renal cell carcinoma and a soft tissue sarcoma.

29. The FGFR inhibitor for use in a method according to claim 27, wherein the FGFR is selected from FGFR1, FGFR2, FGFR3 and, or FGFR4, and said inhibitor is a small molecule inhibitor, an antibody, a ligand trap, a peptide fragment or a nucleic acid inhibitor.

30.-31. (canceled)

32. The method according to claim 27, wherein the FGFR inhibitor selected from NVP-BGJ398, AZD4547, TKI258, JNJ42756493, lucitanib and ponatinib.

33. The method according to claim 27, wherein the method further comprises administering a PDGFRα inhibitor.

34. (canceled)

35. The method according to claim 27, further comprising determining that the cancer is pazopanib resistant and selecting the individual for treatment.

36.-42. (canceled)
